(19) United States
(12) Patent Application Publication
(10) Pub. No.: US 2021/0025887 A1
(43) Pub. Date: Jan. 28, 2021

(54) ANTIBODY OR ANTIBODY COMBINATION AND METHOD USING SAME FOR DETECTION OF AN ANTIGEN RELATED TO MYCOBACTERIUM IN A URINE SAMPLE OF A SUBJECT

(71) Applicant: FOUNDATION OF INNOVATIVE NEW DIAGNOSTICS, Geneva (CH)

(72) Inventor: Tobias BROGER, Geneva (CH)

(21) Appl. No.: 16/981,229

(22) PCT Filed: Mar. 29, 2019
(86) PCT No.: PCT/IB2019/052597

§ 371 Date: Sep. 15, 2020

Related U.S. Application Data
(60) Provisional application No. 62/649,688, filed on Mar. 29, 2018.

(51) Int. Cl. G01N 33/569; C07K 16/12; C07K 16/44

(52) U.S. Cl. CPC G01N 33/5695; C07K 16/1289; C07K 16/44; G01N 2800/52; C07K 2317/92; G01N 2800/26; C07K 2317/33

(57) ABSTRACT

An antibody for the detection of an antigen associated with mycobacteria in an in vitro sample urine of a subject, wherein said antigen comprises Man LAM (Mannose capped Lipoarabinomannan), said antibody specifically binding to said Man LAM molecules from said urine, wherein said antibody binds to said Man LAM with an affinity having a KD of 3×10⁻⁸ M or less, and wherein said antibody binds to LAM molecules that are not capped or that are capped with inositol phosphate with an affinity having a KD of 10 ⁻³ M or more; and an antibody for use of same.

### FIELD OF THE INVENTION

The present invention, in at least some embodiments, relates to an antibody or antibody combination and method using same for detection of an antigen related to mycobacterium in a urine sample of a subject, and in particular, to such an antibody or antibody combination and method which is able to differentiate between disease-causing mycobacterium and other bacterial species with a high degree of accuracy.

### BACKGROUND OF THE INVENTION

Tuberculosis (TB) is the number one infectious disease killer. In 2016, 10.4 million people fell ill with TB and 1.7 million died from the disease making it the ninth leading cause of death worldwide and the leading cause from an infectious agent (World Health Organization 2017). TB is also the most common cause of mortality in people living with HIV (PLHIV), with an estimated 374,000 deaths in 2016 (World Health Organization 2017). The risk of developing TB is estimated to be ˜30 times greater in PLHIV than in people without HIV (Getahun & Ford 2016). Most of the deaths from TB could have been prevented with early diagnosis, however, TB often goes undiagnosed. Globally there was an estimated 4.1 million case gap between estimated incident and reported TB cases (World Health Organization 2017). This gap is due to the limitations of established tests and a lack of accurate, cheap and rapid tests suitable for the typical primary care settings in low and middle income countries (LMICs) where TB is prevalent.

Traditional diagnostic methods are slow (sputum culture) or insensitive (sputum smear microscopy). Modern techniques, such as sequencing or the Xpert® MTB/RIF real time PCR test, have become more available, but require specialized facilities, are costly, or are otherwise inaccessible to many of the populations in LMICs that are most at risk of contracting TB (Pai et al. 2016). The limitations of existing TB diagnostics are especially acute for PLHIV because of the high mortality associated with untreated TB in this population. In addition, while most tests are based on the analysis of sputum samples, many PLHIV with TB have extrapulmonary TB (˜25%) or are otherwise unable to produce sputum samples. A meta-analysis of Xpert clinical performance studies (Steingart et al. 2014) indicated that the pooled sensitivity for HIV-positive patients was lower compared to HIV-negative patients (99% vs. 86%). This result was at least partially explained by the higher proportion of smear-negativity in HIV-positive patients combined with poorer pooled sensitivity of the Xpert test in smear-negative sputum relative to smear-positive sputum (67% vs. 98%). In one cited study, Xpert was only 43% sensitive for detecting smear-negative TB in HIV-positive patients (Lawn et al. 2011). While the sensitivity of the recently developed Xpert Ultra was superior to that of Xpert in patients with HIV, the improved sensitivity came at the expense of a decrease in specificity (Dorman et al. 2017).

Early detection and treatment of all TB patients is a key component of the WHO's End TB Strategy and a requirement to meet the third Sustainable Development Goal (Uplekar et al. 2015), but achieving this will require new sensitive point-of-care (POC) approaches to diagnose active TB in settings such as local health posts or clinics where patients first seek care. The absence of an accurate POC test that can quickly inform treatment decisions leads to significant patient losses at the initial stage of the care cascade, which leads to increased morbidity and mortality (Cazabon et al. 2017). Therefore, the WHO lists a “rapid biomarker-based, non-sputum-based test to detect TB” as a critical unmet need in a high-priority target product profile (TPP) report (World Health Organization 2014).

There has been considerable interest in identifying mycobacterial antigens that are present in the serum or urine of patients with active TB. These antigens represent potential targets for diagnostic tests that would not require the collection of sputum samples and that could be developed in low cost immunoassay-based rapid test formats. Lipoarabinomannan (LAM), a Mycobacterium tuberculosis (Mtb) cell wall lipopolysaccharide and virulence factor, has attracted the most attention as a diagnostic biomarker for TB. LAM has many attractive features as a biomarker: it is bacterially derived, abundant in the cell wall of Mtb, heat stable, and has structural epitopes that are unique to Mtb. There is extensive evidence that LAM can be found in the urine of many TB patients (Sarkar et al. 2014) and earlier-stage studies indicate that it may also be found in sputum (Pereira Arias-Bouda et al. 2000; Cho et al. 1990) and blood (Crawford et al. 2017; Sada et al. 1992).

Since the first reports of LAM in sputum in 1990 (Cho et al. 1990), in blood in 1992 (Sada et al. 1992) and urine in 1997 (Svenson 1997), the attractive features of LAM as a diagnostic target have inspired a quarter of a century of research on LAM-based diagnostics including the development of commercial ELISA and rapid (lateral flow) tests. The only LAM test currently on the market for clinical diagnosis of TB, the Alere Determine TB LAM® test (LF-LAM) from Abbott Diagnostics, is a lateral flow test for measuring LAM in urine that uses rabbit polyclonal antibodies. Another test, an ELISA test from Otsuka Pharmaceutical for measuring LAM in sputum, recently received a CE mark for use in Europe, but is only being used as a tool for evaluating the response to treatment in TB drug trials (Kawasaki et al. 2018).

Despite strong initial excitement after the introduction of kits for measuring LAM in urine and numerous clinical studies evaluating their performance, the adoption of these tests has been limited. The main reason for the low adoption has been the relatively poor clinical sensitivity of the tests across the spectrum of incident TB cases. A comprehensive meta-analysis of studies evaluating the Alere LF-LAM test for diagnosing TB in PLHIV found the test to have limited diagnostic accuracy: 44% pooled sensitivity for TB diagnosis in HIV+cases at 92% pooled specificity in 3037 patients (World Health Organization 2015). This analysis formed the basis of the very limited WHO recommendation to only use the Alere LF-LAM to diagnose TB in immunocompromised people living with HIV/AIDS (PLHIV) with CD4 counts ≤100 cells/μl and with TB symptoms (World Health Organization 2015). Despite its low sensitivity, testing with Alere LF-LAM, followed by immediate treatment was shown to significantly reduce 8-week mortality in a large, pragmatic, randomized, parallel-group, multicentre trial in HIV+inpatients from ten African hospitals (Peter et al. 2016), but so far only the Central African Republic and Zimbabwe have guidelines on how to integrate the Alere LF-LAM in diagnostic testing and there has been very limited uptake in LMIC's (MSF & Stop-TB Partnership 2017).

Despite the numerous studies that have been conducted with the LF-LAM test and earlier ELISA tests, there are major unanswered questions about urinary LAM as a TB biomarker that are critical to understanding whether there is path to improving the clinical performance of LAM tests. Most importantly, it is not understood whether the LAM levels in the urine of LF-LAM-negative TB patients—especially HIV− patients and HIV+ patients with high CD4 counts—are simply too low to be measured by the current test, but could be measured by a test with improved detection limits. Secondly the availability and abundance of the most relevant and Mtb-characteristic LAM epitopes in urine is poorly understood.

### BRIEF SUMMARY OF THE INVENTION

Current diagnostic tests for tuberculosis (TB) based on the measurement of mycobacterial lipoarabinomannan (LAM) in urine have many desirable attributes for use in low and middle income countries, such as low-cost and ease-of-use, but are insufficiently sensitive. However, urine based tests with higher accuracy (i.e. with higher clinical sensitivity) would clearly be desirable.

The present invention overcomes these drawbacks of the background art by providing an antibody or antibody combination and method using same for detection of an antigen related to mycobacterium in a urine sample of a subject. The antibody or antibody combination and method is able to differentiate between disease-causing mycobacterium and other bacterial species with a high degree of accuracy.

According to at least some embodiments, there is provided an antibody for the detection of an antigen associated with mycobacteria in an in vitro sample urine of a subject, wherein said antigen comprises ManLAM (Mannose capped Lipoarabinomannan), said antibody specifically binding to said ManLAM molecules from said urine, wherein said antibody binds to said ManLAM with an affinity having a KD of 3×10⁻⁸ M or less, and wherein said antibody binds to LAM molecules that are not capped or that are capped with inositol phosphate with an affinity having a KD of 10⁻³ M or more.

Optionally, the ManLAM comprises MTX-capped ManLAM, characterized in that the mannoside caps are further modified by attachment of a 5-deoxy-5-methylthio-xylo moiety.

Optionally, the MTX-capped ManLAM comprises MTX-Man2-capped ManLAM characterized by two alpha 1-2-Manp-linked residues that are further substituted with an alpha 1-4-linked methylxylose residue.

Optionally, the antibody binds to an epitope of said ManLAM comprising a Manp feature.

Optionally, the antibody binds to an epitope of said ManLAM characterized as featuring a motif selected from the group consisting of Glycan7, Glycan8, Glycan9, Glycan 10, and Glycan11.

Optionally, the epitope is further characterized as featuring a MTX-dimannose portion.

Optionally, the antibody is suitable for detecting a presence of a slow-growing mycobacteria in a subject using a sample of the urine from the subject.

Optionally, the antibody is suitable for detecting the antigen associated with Mycobacterium tuberculosis or M. bovis.

According to at least some embodiments, the antibody is able to specifically detect an antigen associated with disease-causing mycobacteria, and to distinguish markers associated with such bacteria from other types of bacteria.

Optionally, the antibody does not cross react with a marker from fast growing mycobacteria in the urine of the subject.

Optionally, the antibody does not cross react with a marker associated with M. fortuitum, M. smegmatis M. abscessus, or M. chelonae.

Optionally, the antibody shows at least 10 fold lower reactivity to a marker associated with a slow-growing mycobacteria selected from the group consisting of M. gordonae. M. intracellulare, and M. avium.

Optionally, the antibody detects the antigen associated with said Mycobacterium tuberculosis or M. bovis with at least 1500 fold greater reactivity in comparison to detection of non-mycobacteria bacterial species.

Optionally, the non-mycobacterium bacterial species comprises one or more of Gordonia bronchialis, Nocardia asteroids, Rhodococcus sp., Tsukamurella paurometabolum, Candida albicans, Corynebacterium urealyticum, Escherichia coli, Klebsiella pneumoniae, Streptococcus agalactiae, Staphylococcus saprophyticus, Pseudomonas aeruginosa, Staphylococcus aureus, Proteus mirabilis, Proteus vulgaris, Neisseria gonorrhoeae, Haemophilus influenza, Enterococcus faecalis, Enterobacter aerogenes, or Chlamydia trachomatis.

According to at least some embodiments, the antibody may comprise a plurality or combination of antibodies, preferably used in an immunoassay. More preferably the immunoassay is a sandwich immunoassay, in which one antibody “captures” the antigen while a second antibody detects the presence of the captured antigen. Each antibody binds to a different epitope on the antigen, to avoid competing each other off of the antigen. The detection antibody may have a lower binding affinity for the antigen than the capture antibody.

According to at least some embodiments, the antibody as described herein is suitable for use as a capture antibody in a sandwich immunoassay for detecting the antigen.

Optionally the antibody is suitable for use as a detection antibody in a sandwich immunoassay for detecting the antigen.

According to at least some embodiments, there is provided a method for differentially detecting a presence of disease-causing mycobacteria in a subject, comprising contacting an antibody according to any of the above claims with the urine of the subject; detecting binding of said antibody to an antigen in the urine; if said antibody binds specifically to said antigen in the urine with an affinity having a KD of 3×10⁻⁸ M or less, determining that said disease-causing mycobacteria characterized by said ManLAM molecules is present in the subject's body.

Optionally the antibody binds to an antigen of said disease-causing mycobacteria in the urine with a signal at least three times greater than to an antigen of non-disease causing mycobacteria.

Optionally the method further comprises applying a first antibody to the urine, said first antibody being characterized according to any of the above claims, to bind to said antigen; and applying a second antibody to the urine to bind to a second antigen, wherein said second antibody does not bind to the same antigen as the first antibody, and wherein said first and second antigens comprise said ManLAM molecules; wherein one of said first and second antibodies is a capture antibody and wherein the other of said first and second antibodies is a detection antibody in an immunoassay.

Optionally the second antibody is characterized as binding to poly-arabinose structures of said ManLAM molecules with an affinity having a KD of 3×10−5 M or less.

Optionally the antibody binds specifically to ara4 and/or ara6.

Optionally the method further comprises contacting the urine with an antibody selected from the group consisting of MoAb1, 13H3, 27D2 and A194-01 antibodies. The MoAb1 antibody is described in U.S. Pat. No. 9,512,206, incorporated by reference as if fully set forth herein to the extent necessary to support the claims. The A194-01 antibody is described in US Patent Application Publication No. US2017016058, incorporated by reference as if fully set forth herein to the extent necessary to support the claims.

Optionally the method further comprises contacting the urine with a combination of a plurality of the MoAb1, 13H3, 27D2 or A194-1 antibodies in a sandwich immunoassay.

Optionally the method further comprises contacting the urine with a combination of the MoAb1 and A194-1 antibodies in a sandwich immunoassay.

Optionally the method further comprises applying the MoAb1 antibody to a sample with a suitable second antibody to achieve a fold change of 3 or greater between median signals of samples from subjects suffering from tuberculosis compared to samples from subjects without tuberculosis using a suitable reference standard diagnosis for classification of the subjects.

Optionally the reference standard diagnosis is based on mycobacterial culture or PCR based methods to classify subjects.

Optionally the method further comprises applying the MoAb1 antibody to a sample to detect at least 20% more subjects suffering from tuberculosis compared to samples from subjects without tuberculosis using a suitable comparative standard assay, wherein said suitable comparative standard assay comprises the Alere LF-LAM.

Optionally the detected ManLAM antigen specific sandwich immunoassay signal in a urine sample from a subject without tuberculosis is below 11 pg ManLAM/ml for at least 70% of the samples in a population.

Optionally the signal is below the limit of detection for at least 80% of the samples in the population.

Optionally the signal is below the limit of detection for at least 90% of the samples in the population.

Optionally the signal is below the limit of detection for at least 95% of the samples in the population.

Optionally the signal is below the limit of detection for at least 97% of the samples in the population.

Optionally the detected ManLAM antigen specific sandwich immunoassay signal in a urine sample from a subject with tuberculosis is above 11 pg ManLAM/ml for at least 40% of the samples in a population.

Optionally the signal is above the limit of detection for at least 50% of the samples in the population.

Optionally the signal is above the limit of detection for at least 60% of the samples in the population.

Optionally the signal is above the limit of detection for at least 75% of the samples in the population.

Optionally the signal is above the limit of detection for at least 90% of the samples in the population.

Optionally the method further comprises detecting TB disease-causing mycobacteria in the subject in the absence of the HIV virus.

Optionally an AUC (area under the curve) of an immunoassay based on binding of said antibodies to said antigen for binary diagnostic classification of subject suffering from tuberculosis versus subjects without tuberculosis is at least 0.70.

Optionally the AUC is at least 0.80.

Optionally the AUC is at least 0.85.

Optionally the AUC is at least 0.90.

Optionally the AUC is at least 0.95.

Optionally the AUC is at least 0.98.

Optionally the method further comprises applying a combination of the MoAb1 antibody or the 13H3 antibody as the first antibody, and the A194-01 antibody or the 27D2 antibody as the second antibody to detect an antigen associated with mycobacteria in an in vitro urine sample from a subject, in an immunoassay in which one of the first and second antibodies is the capture antibody and the other of the first and second antibodies is the detection antibody.

Optionally the detection is performed by using an immunoassay, wherein the combination has at least 20% higher clinical sensitivity than the Alere LF-LAM test.

Optionally the method further comprises diagnosing the subject with tuberculosis according to a presence of said disease-causing mycobacteria in the body of the subject.

Optionally the diagnosing further comprises detecting a presence of an active tubercular infection in the subject.

Optionally the method further comprises monitoring efficacy of treatment of the subject for tuberculosis according to the presence of said disease-causing mycobacteria.

Optionally the method further comprises concentrating said antigen comprising ManLAM in the sample prior to detection with the immunoassay to further increase clinical sensitivity.

Optionally the concentrating of said antigen comprises applying magnetic beads or ultrafiltration to the sample.

Optionally the method further comprises differentiating between a presence of a disease-causing mycobacteria in the subject and a non-disease causing mycobacteria in the subject.

Optionally the method further comprises specifically detecting a presence of a disease-causing mycobacteria in the subject in a presence of contaminating bacteria from an environment of the subject.

Optionally the contaminating bacteria comprise one or more of Gordonia bronchialis, Nocardia asteroids, Rhodococcus sp., Tsukamurella paurometabolum, Candida albicans, Corynebacterium urealyticum, Escherichia coli, Klebsiella pneumoniae, Streptococcus agalactiae, Staphylococcus saprophyticus, Pseudomonas aeruginosa, Staphylococcus aureus, Proteus mirabilis, Proteus vulgaris, Neisseria gonorrhoeae, Haemophilus influenza, Enterococcus faecalis, Enterobacter aerogenes, or Chlamydia trachomatis, or Nontuberculous mycobacteria.

Optionally the method further comprises heating the urine before contacting said antibody.

According to at least some embodiments, the HIV status of the subject does not significantly impact on the ability of the antibody combination and method to determine whether the subject is suffering from an infection by disease causing mycobacteria. An antigen comprising Lipoarabinomannan (LAM) is detectable in the urine of HIV positive and HIV negative tuberculosis (TB) subjects.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-C. Results of screen to identify antibody pairs for detecting LAM. (A) Schematic of the sandwich immunoassay used for screening and measurements. (B) Heat maps that show the ability of each pairwise combination of capture (rows) and detection (columns) antibodies to detect 10 ng/mL of purified LAM from cultured Mtb (left heat map) and LAM in the urine from TB-positive, HIV-positive individuals (right heat map). The heat maps display the signal to blank (S/B) ratio. The value in the urinary LAM heat map represents the maximum value for urine samples from two individuals. The antibody names are color coded based on the LAM epitopes they target, as determined by binding to glycan arrays and the epitopes are listed next to the names of the capture antibodies (see FIG. 2B and FIG. 7 for details of the epitope mapping results). Antibody combinations that show high reactivity with purified LAM and LAM in urine from TB-positive, HIV-positive individuals are indicated with a red box. (C) Schematic of LAM illustrating the different epitopes listed in the heat map.

FIGS. 1D-1E show the structure of 61 oligosaccharide structures that were used for antibody epitope mapping. Selected oligosaccharides (Gly 16, Gly 22, and Gly 44) were further used for the development of rabbit monoclonal antibodies. The key is shown in FIG. 1F.

FIG. 2A shows immunoassay signals as a function of LAM concentration for the best performing anti-LAM antibodies; MoAb1 as a capture antibody combined with the A194-01 as a detection antibody. The points show the measured immunoassay signal when testing blank samples (n=10) and 7 levels of LAM (n=4 per level). Solid lines show the 4PL fit to the data. The dashed lines show the signal that gives an S/B ratio of 1.375 and the associated LAM limit of detection (LOD) calculated from the fitted curves for both pairs.

FIG. 2B shows the results of epitope mapping using glycan arrays. Reactivity of monoclonal antibodies at a concentration of 0.039 μg/mL to selected oligosaccharide structures. Dark green areas represent strong binding, white areas no- or low blinding. The figure includes all glycans to which reactivity over background was shown for at least one antibody. The naming in column one refers to FIGS. 1D-1E.

FIG. 3. (A) Heat map showing the measured LAM concentrations for all tested urine samples (table columns) for five capture antibodies when paired with the A194-01 detection antibody. The samples are grouped by the donors TB and HIV status. The bottom row of the table provides the Alere LF-LAM test grade for each sample for comparison (only samples with positive Alere LF-LAM test results are colored). (B) The result from FIG. 3A for the sandwich immunoassay using the MoAb1 capture/A194-01 detection antibody pair are replotted in scatter plot format. The plot shows the measured signal to blank (S/B) ratios (left axis) and LAM concentrations (right axis) for each urine sample as a function of the TB and HIV status of the donor. The dashed orange line shows the assay threshold (S/B=1.375). Concentration values are only meaningful for points above the assay threshold. The points are colored by the results of the Alere LF-LAM test for the same samples. Scatter plots for the other 3 capture antibodies combined with 194-01 as a detection antibody can be found in FIG. 3C. FIG. 3C shows the measured signal to blank (S/B) ratios (left axis) and LAM concentrations (right axis) for each urine sample as a function of the TB and HIV status of the donor. Each plot shows the results for one of the 3 capture antibodies in the capture antibody panel when paired with the A194-01 detection antibody. The dashed orange line shows the assay threshold (S/B=1.375). Concentration values are only meaningful for points above the assay threshold. The points are colored by the results of the Alere LF-LAM test for the same samples.

FIG. 4 shows a plot of the measured concentration of LAM for a set of four urine samples from TB+HIV+ patients using the MoAb1 capture/A194-01 detection antibody combination. The light bars were the measured concentrations when samples were tested without pretreatment. The dark bars represent results when the samples were heat treated (85° C. for 10 minutes) prior to testing.

FIG. 5. (A-B) Assays signals for TB+ subjects broken down by (b) HIV status and CD4 count (in cells per μL) and (C) Alere LF-LAM test. Asterisks indicated significant differences from the left-most condition (Mann-Whitney test, p<0.05).

FIG. 6. Plot shows the observed clinical sensitivity and specificity (with 95% confidence intervals) for each candidate capture antibody when paired with the A194-01 detection antibody. The plot also shows the minimal (triangle) and optimal (diamond) target sensitivity and specificity requirements set by the WHO in its target product profile (TPP) requirements document for POC TB tests used for two different use case scenarios: (i) definitive detection/diagnosis of TB (purple symbols) or (ii) triage to identify patients who should undergo further confirmatory testing for TB (green symbols). The marker representing the performance of an assay would ideally be above and to the left of the marker representing the requirement for a use case (the area of interest is highlighted). 95% confidence Wilson confidence intervals are indicated.

FIG. 7 shows the results of epitope mapping using glycan arrays. Reactivity of monoclonal antibodies at six different antibody concentrations to all 61 structures from FIGS. 1D-1F and three negative control spots (Two times BSA and Buffer) and one positive control spot (LS).

FIG. 8: Forest plots of sensitivity and specificity and differences between Antibodies Under Test and Alere LF-LAM against the microbiological and composite reference standards. (A) All cohorts combined using a bivariate random-effect model for analysis, (B) for the three cohorts separately, and (C) for the pooled analysis using a bivariate random-effect model in three CD4 strata. *Sensitivity and specificity estimates based on analysis using a bivariate random-effect model are indicated with an asterisk. MRS denotes microbiological reference standard, CRS composite reference standard, ΔSn sensitivity difference, ΔSp specificity difference, HIV human immunodeficiency virus and CI confidence interval.

FIG. 9: Venn diagrams. Proportions of (A) all microbiologically confirmed TB diagnoses (n=141), and (B) microbiologically confirmed TB diagnoses in patients with CD4≤100 cells/μl (n=74), which could be made by performing a single urine Antibodies Under Test measurement, urine Alere LF-LAM, urine Xpert, sputum Xpert or sputum smear microscopy test on samples obtained within 24 hours of admission for Cohort2. The tables below the Venn diagrams report the diagnostic yield per test method. “TB cases missed by above methods” include those made by positive mycobacterial culture on any specimen collected at any point during patient admission and/or diagnoses made on the basis of Xpert testing on any specimen collected after the first 24 hours. Numbers embedded within the Venn diagram represent the number of TB cases diagnosed by a given assay or assays.

### DESCRIPTION OF AT LEAST SOME EMBODIMENTS

LAM assays for detection of antigen(s) in urine of a subject associated with disease causing mycobacteria are highly desirable, because such tests would be inexpensive and easy to administer, even under challenging medical and clinical conditions. The present invention, in at least some embodiments, relates to improved assay reagents and methods, to provide sensitive immunoassays for LAM using antibodies targeting a variety of LAM epitopes.

Overall 8 candidate antibodies for LAM that were developed using different approaches and that targeted a variety of different LAM motifs were evaluated. These antibodies were screened to identify the antibody pairs that provided the best analytical sensitivity for LAM detection in a sandwich format. The best candidate pairs were then evaluated in a retrospective case-control study of 75 urine samples from HIV+ and HIV− adults presenting at primary care sites with clinical symptoms of TB. The cross-reactivity of the candidate pairs was also evaluated for potentially interfering non-TB bacteria and microorganisms.

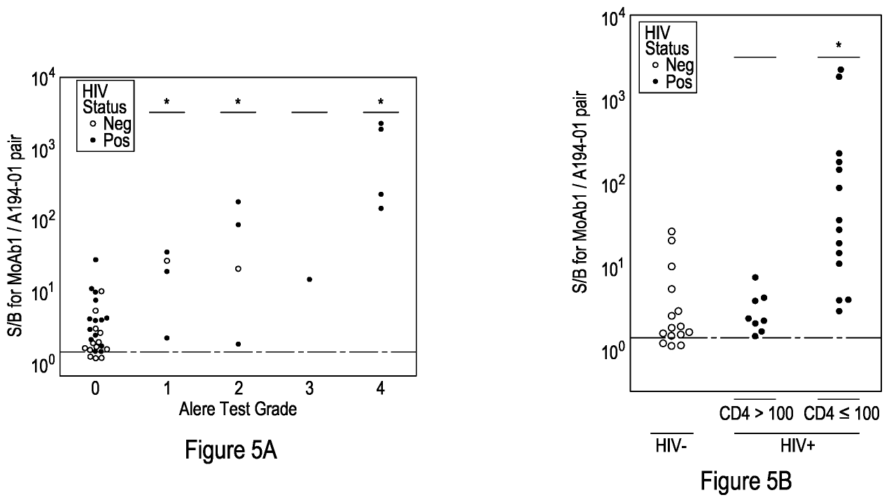

Without wishing to be limited in any way, one aspect of the present invention relates to an antibody pair that demonstrated, in the immunoassay format, excellent sensitivity [93% (CI: 80%-97%)] and specificity [97% (CI: 85%-100%)] across the full sample set (FIG. 5). Importantly, the assay showed high sensitivity [80% (CI: 55%-93%)] even when analysis was limited to the HIV− subjects. By contrast, the commercial Alere LF-LAM strip test for LAM showed an overall sensitivity of 33% (CI: 20%-48%) and a sensitivity for the HIV− subpopulation of only 13% (CI: 4%-38%). The selected pair used a capture antibody that targets the methylthio-d-xylose (MTX) structure, which is relatively specific to LAM from TB-causing mycobacteria; no cross-reactivity was observed for fast-growing mycobacteria or for LAM-producing non-mycobacterial actinomycetes, other common urinary tract infections or potentially cross-reacting cells (Table 2A). This specificity appears to be important, as another less TB-specific capture antibody provided higher analytical sensitivity, but poorer clinical specificity.

### EXAMPLE 1

### LAM-BAsed Assays for Detection of an Antigen Associated with Disease Causing Mycobacteria in Urine

This Example relates to an illustrative LAM-based assay for detection of one or more antigens in urine associated with disease causing mycobacteria.

Materials and Methods

Antibodies and control materials

Purified LAM from Mtb strain Aoyama-B was obtained from Nacalai USA, Inc. (San Diego, USA). Phosphoinosotiol-capped LAM (PILAM) from M. smegmatis and inactive whole cell lysates of Mtb and M. bovis were obtained from the Biodefense and Emerging Infections Research Resources Repository (BEI, Manassas, USA). For cross-reactivity testing, live whole cell stocks of a number of different bacterial and fungal strains were obtained from ATCC as vials of lyophilized cells or frozen cell suspensions in glycerol. The lyophilized cells were suspended in 0.5 mL of 2% bovine serum albumin (BSA) in phosphate buffered saline (PBS). The stock cell suspensions were then serially diluted in the same buffer to create test samples.

A number of existing monoclonal and polyclonal anti-LAM antibodies were graciously provided by collaborating academic and commercial groups including Dr. Abe Pinter/Rutgers (A194-01) (Choudhary et al. 2018; Kaur et al. 2002; US Patent Application Publication No. US2017016058). A commercial anti-LAM rabbit polyclonal antibody (Viro Poly) was purchased from Virostat, Inc. (Westbrook, USA).

MoAb1, MoAb2, MoAb3 are recombinant antibodies isolated at Otsuka Pharmaceutical by phage display of ScFv libraries generated from chickens (MoAb2) and rabbits (MoAb1 and MoAb3) immunized with BCG and panned against ManLAM (U.S. Pat. No. 9,512,206). These antibodies were expressed by synthesizing the variable region sequences and by inserting them into standard IgG1 vectors and transfecting the plasmids into Expi293 cells.

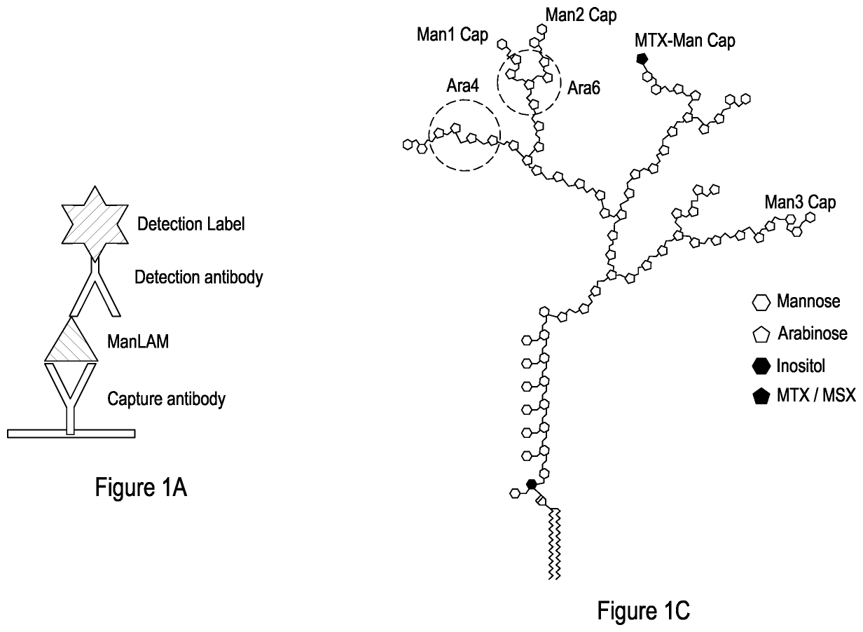

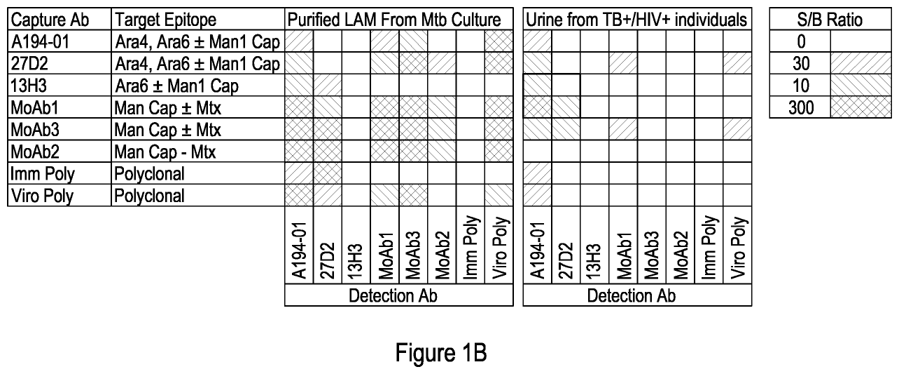

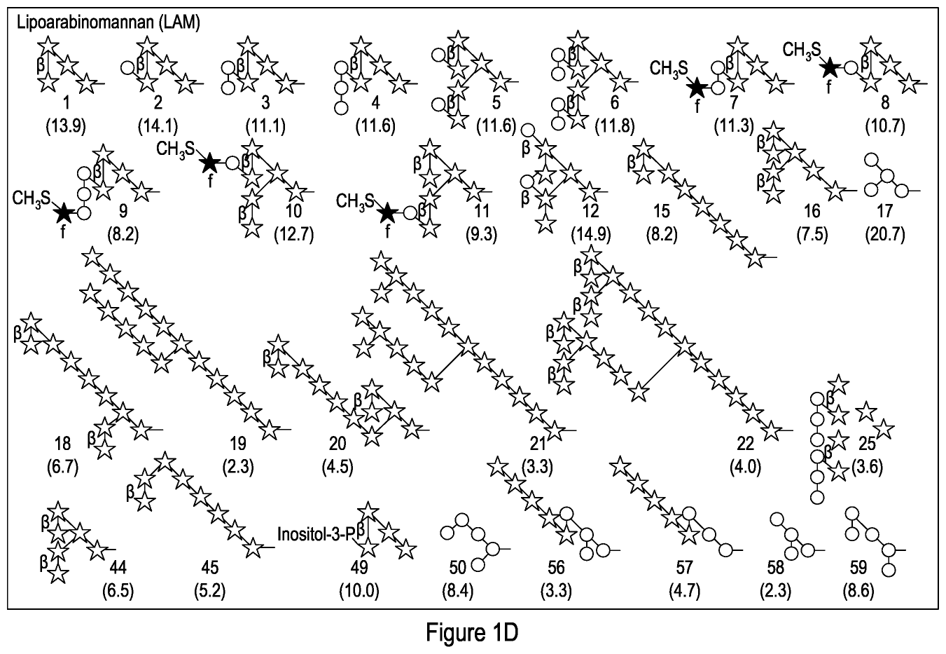

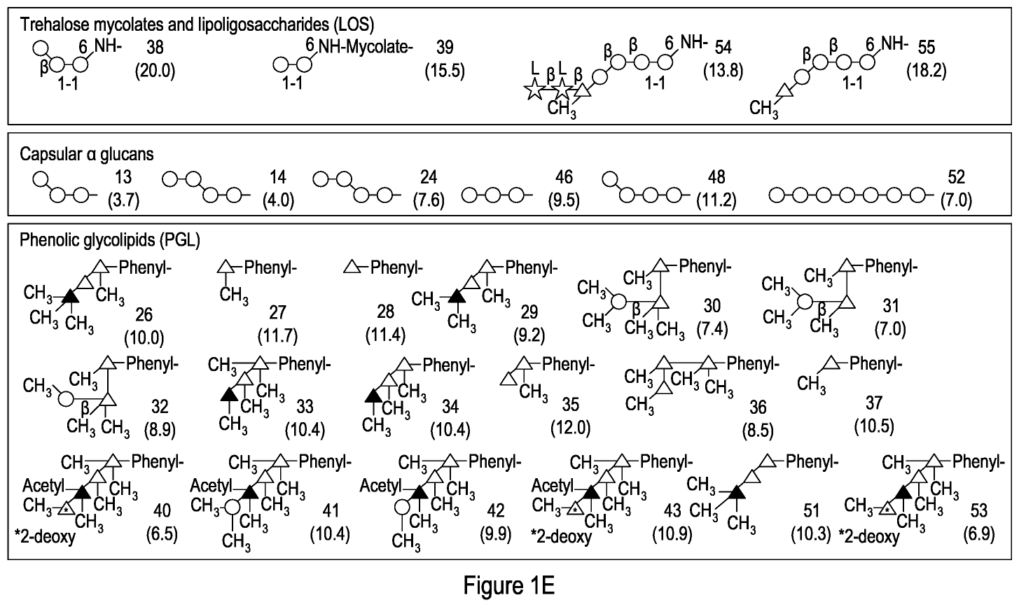

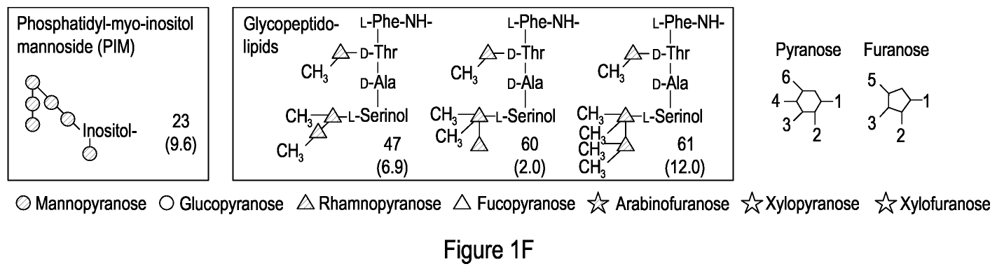

In addition, two recombinant rabbit monoclonal antibodies (13H3 and 27D2) and a rabbit polyclonal antibody (Imm Poly) were newly produced. The monoclonal antibodies were generated using synthetic LAM oligosaccharide fragments coupled to bovine serum albumin (provided by Dr. Todd Lowary, University of Alberta) as the immunogen. Briefly, a rabbit was immunized with 65 μg BSA-Ara6 (ID 44), 65 μg BSA-Aral (ID 16) and 65 μg BSA-Ara22 (ID 22) and boosted with the same mixture on days 7 and 14 (see FIG. 1D) for a description of the oligosaccharide structures). Peripheral blood mononuclear cells (PBMCs) were collected at day 28 and cultured in 96 well plates. Supernatants were tested by indirect ELISA for binding to LAM and low cross-reactivity to BSA. The antibody genes from wells with the desired activity were cloned, expressed in HEK293 cells and tested by indirect ELISA to identify the antibodies (13H3 and 27D2) with the most reactivity to purified LAM and heat-killed Mtb H37Ra. The polyclonal antibody (Imm Poly) was produced by immunizing a rabbit with a mixture of 250 μg purified LAM, 200 μg heat killed Mtb H37Ra and Incomplete Freunds Adjuvant (ICFA) and boosting with the same immunogen at days 28, 47 and 67. Serum collected on day 76 was purified by affinity chromatography on a Protein A column.

13H3 has the following translated variable region sequence:

13H3 Heavy chain (the underlined sequence indicates
the start of the constant region):
METGLRWLLLVAVLKGVQCQSVEESGGRLVTPGTPLTLTCTASGSDIFTYY
MNWVRQARGKGLEWIGYINTGGSAWYSSWAKGRFTISRTSTTVTLKMTSPT
TEDTATYFCARAIGGGAAGGDLWGQGTLVTVSSGQPKAPSVFPLAP
13H3 Kappa chain: (the underlined sequence
indicates the start of the constant region)
MDTRAPTQLLGLLLLWLPGATFAIVMTQTPSSKSVPVGDTVTINCQASESV
ANNNDLAWYQQKPGHSPKLLIYFASTLASGVPSRFKGSGFGTQFTLTISGA
QCDDAATYYCTGYKGFNTDGMAFGGGIEVVVKGDPVAPTVLIFPPAA

27D2 has the following translated variable region sequence: 11H3/11K2

27D2 Heavy chain (the underlined sequence indicates
the start of the constant region):
METGLRWLLLVAVLKGVQCQSVEEFGGRLVTPGTPLTLTCTVSGFSLSRKW
MSWVRQAPGKGLEWIGIITDSGSTYYASWVNGRFTISKTSTTVTLKITSPS
TEDTATYFCGKDDYIYGDLWGSGTLVTVSSGQPKAPSVFPLAP
27D2 Kappa chain: (the underlined sequence
indicates the start of the constant region)
MDTRAPTQLLGLLLLWLPGATFAQVLTQTPASVSAAVGGTVTISCQSSQSV
YNNNWLAWYQQKPGQPLKQLIYAASSLASGVPSRFKGSGSGTQFTLTISDL
ECDDAATYYCQGGYTTHARAFGGGTEVVVKGDPVAPTVLIFPPAA

All antibodies were analyzed to identify the oligosaccharide epitopes recognized by their binding sites. Epitope identification was carried out by measuring the binding of the antibodies to glycan arrays presenting a diverse set of 61 oligosaccharide structures as described in (Choudhary et al. 2018). The set of structures is shown in FIGS. 1D-1F. Briefly, oligosaccharide fragments were synthesized as previously described (Gadikota et al. 2003; Joe et al. 2006; Joe et al. 2007; Sahloul & Lowary 2015) conjugated to BSA and used to generate microarrays. Serial dilutions of antibodies were incubated on the slides for 30 minutes at 37° C., and after washing, for 40 minutes stained with fluorescently labeled secondary anti-species antibodies. Florescence signals were measured using a GenePix 4000B scanner (Molecular Devices, Sunnyvale, USA) and intensity of each spot was quantified using ProMicroarray Image Analysis Software 6.1.

LAM Immunoassays

Immunoassays for LAM employing a multiplexed sandwich immunoassay format (FIG. 1A) and electrochemiluminescence (ECL) detection were carried out on commercial instrumentation and multi-well plate consumables from Meso Scale Diagnostics, LLC. (MSD) (Debad et al. 2004). The assays were run in MSD's U-PLEX® 96-well plates. On the bottom of each well of the plate there is a 10-plex array of binding reagents immobilized on an integrated screen-printed carbon ink electrode. The 10 binding reagents each bind to one of a set of 10 proprietary linkers. In U-PLEX assays, different capture reagents are coupled to different linkers. Arrays of the capture reagents in the plates are formed as needed by adding a mixture of the capture antibody-linker conjugates to the well and allowing the linkers to self-assemble on their complementary array elements (or “spots”). Arrays of anti-LAM antibodies were used to compare the performance of multiple capture antibodies in a single multiplexed measurement.

Antibodies were prepared for use in the assays according to the procedures in the U-PLEX package insert. Capture reagents were biotinylated with Sulfo-NHS-LC-Biotin (Thermo Fisher Scientific) and coupled via biotin-streptavidin binding to U-PLEX linkers. Detection antibodies were labeled with the MSD SULFO-TAGTM ECL label. To prepare the capture antibody arrays, up to 10 antibody-linker conjugates were combined in U-PLEX Stop Buffer at a concentration of 2.9 μg/mL per antibody and 50 μL of this mixture was added to each well of the U-PLEX plates. The plates were incubated for one hour with shaking to allow the antibody arrays to assemble and then washed. The plates were used immediately or stored at 4° C. in a desiccated pouch until needed.

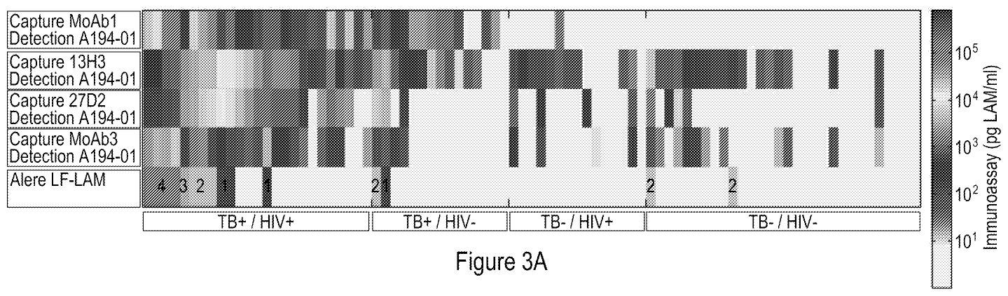

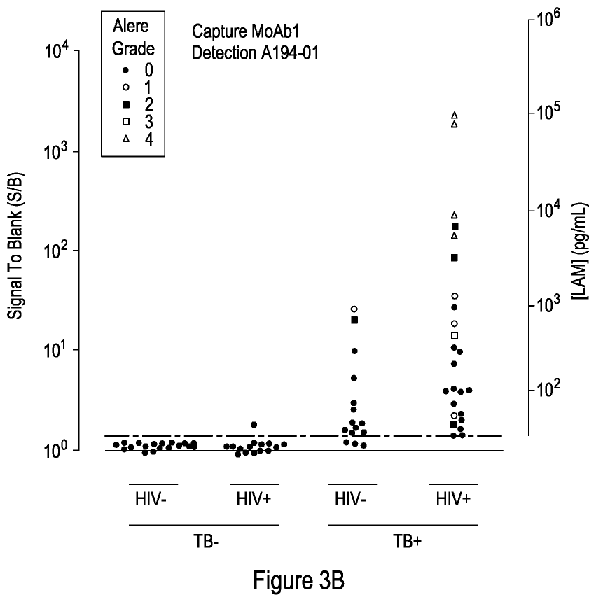

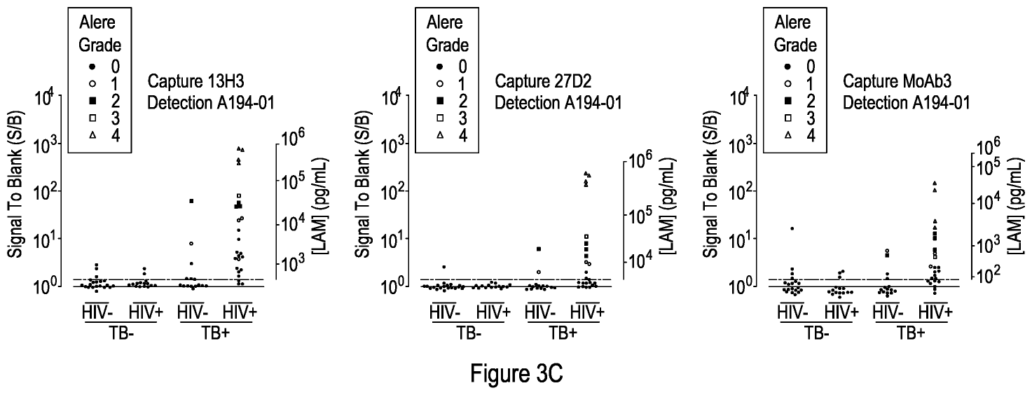

Unless otherwise indicated, assays were run according to the following protocol using commercial diluents from MSD that include blocking components to prevent non-specific signals from human anti-mouse antibodies (HAMAs) or other non-specific antibody binding proteins. Capture antibody arrays were pre-formed in a U-PLEX plate as described above. MSD Diluent 22 (25 μL) was combined with 25 μL of sample in each well of the U-PLEX plate and the mixture was incubated with shaking for 1 hour at room temperature to bind LAM in the sample to the capture antibody array in the well. After washing the wells to remove unbound sample, 25 μL of 2 μg/mL SULFO-TAG labeled detection antibody (in MSD Diluent 3 supplemented with casein) was added and incubated for an additional hour with shaking to complete the immunoassay sandwich. After washing the wells to remove unbound detection antibody, the wells were filled with 150 uL of 2× MSD Read Buffer T and ECL was measured on an MSD Sector® S 600 ECL plate reader. The plate reader applies a voltage to the electrodes in the MSD plates to induce ECL from the bound detection antibodies, and uses a cooled CCD camera to quantitate the light emission from each array spot (Debad et al. 2004). During screening for antibody pairs all possible combinations of capture antibody and detection antibodies A194-01, 27D2, 13H3, MoAb1, MoAb2, MoAb3, Imm Poloy, Viro Poly were evaluated with purified LAM from Mtb culture and Urine from TB+/HIV+ individuals (FIG. 1B). More detailed assay evaluation was carried out on a specific panel that combined an array of capture antibodies (MoAb1, 13H3, 27D2, MoAb3) and the A194-01 or 27D2 detection antibodies (FIG. 3A).

LAM Quantitation

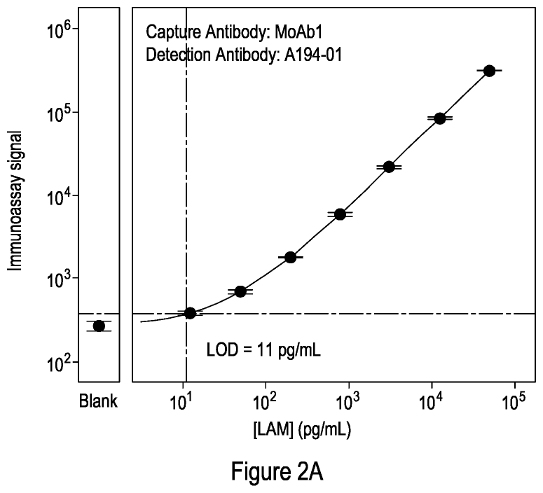

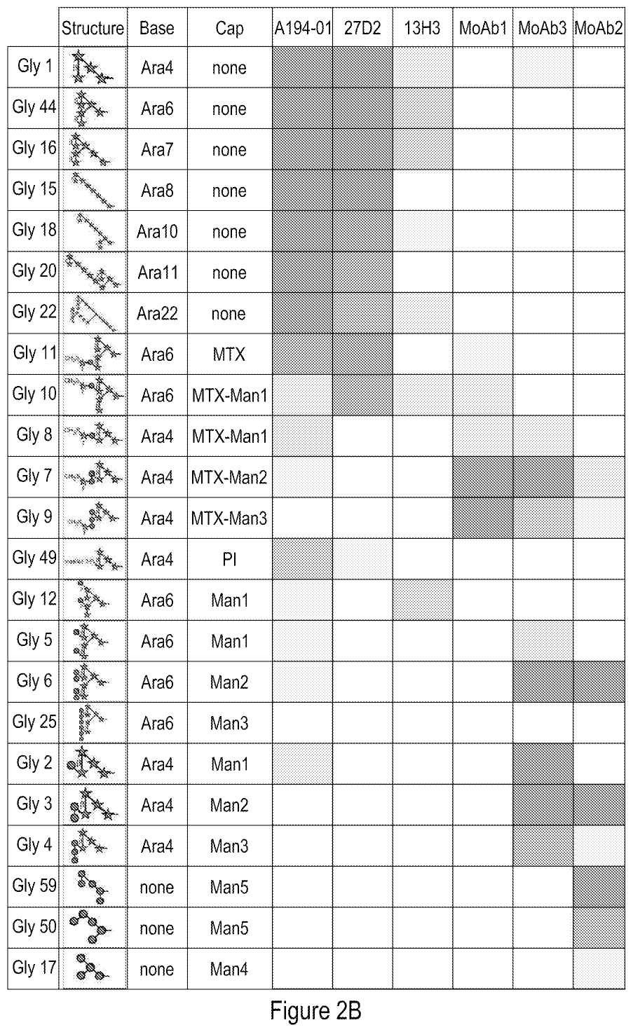

To calculate LAM concentrations, an 8-point calibration curve with purified LAM (diluted in MSD Diluent 100) was run in duplicate in each assay plate. The relationship of ECL signal to LAM concentration was fit to a 4-parameter logistic (4-PL) function. LAM concentrations for test samples were calculated by back-fitting ECL signals to the 4-PL fit. An exemplary calibration curve is shown in FIG. 2A.

Urine Sample Preparation

To inactivate any anti-LAM antibodies, samples were pre-treated prior to analysis by heat treatment at 85° C. for 10 minutes.

Sample collection and diagnostic tests

For this retrospective case-control study, a total of 75 urine samples were selected from FIND'S (Geneva, Switzerland) biobank. These samples were previously collected in studies from adults presenting at primary care sites in Bangladesh (n=5), Peru (n=19), South Africa (n=15) and Vietnam (n=36) with clinical symptoms of TB, but not receiving TB treatment at the time of sample collection. Approval by local Ethics Committee and informed patient consent was obtained before enrolling patients and no personally identifiable information was available to FIND or to the researchers.

FIND uses standardized protocols for collection and processing of samples. Briefly urine was collected at first contact with the patient, processed, aliquoted and frozen (−80° C.) on the same day (typically within 4 hours). WHO prequalified IVD's were used for HIV serological testing and CD4 counting. For use in patient classification, sputum samples (typically two in the first 24 h) were also collected for all participants, decontaminated and cultured up to 6-times using liquid culture (MGIT, BD, Franklin Lakes, USA) and solid culture (Loewenstein-Jensen media). The presence of Mtb complex in cultures was confirmed by either Ziehl-Neelson or Auramine-O florescence Microscopy to identify acid-fast bacilli, MPT64 antigen detection using rapid speciation assays (like the Capilia TB test, TAUNS, Japan) or molecular methods.

Classification of patients and composite reference standard

Patient were classified using a composite reference standard on the basis of clinical and laboratory findings as described elsewhere (Broger et al. 2017). TB-positive (TB+) were patients with at least one positive culture. All TB+ patients had positive microscopy results. Participants who were smear negative and culture negative on ≥4 cultures on all sputum samples and who exhibited symptoms resolution in the absence of tuberculosis treatment and negative sputum culture results at 2-month follow-up visit were classified as TB−. Subjects were further classified as HIV+ or HIV− based on HIV rapid tests (Table 3A, see below).

Rapid Tests for LAM

Urine samples were tested using the Alere LF-LAM test run according to the manufacturer instructions. The strip was read by three different technicians independently who compared the test line intensity with the reference card provided by the manufacturer and graded the results. For documentation, all strips were scanned.

Results

Antibody Generation and Selection

To identify potentially useful antibody pairs for use in sandwich immunoassays, a pre-existing library of antibodies was tested (A194-01, 27D2, 13H3, MoAb1, MoAb2, MoAb3, Imm Poloy, Viro Poly) in every possible pairwise combination of capture and detection antibody. Each pair was then tested to assess its background signal for a blank sample, its specific signal for purified LAM from cultured Mtb, and its specific signal for urinary LAM in urine from two TB-positive HIV-positive human subject known to contain a high level of LAM (based on positivity with the Alere LF-LAM test). To conserve sample, each test well had an array of capture antibodies, but was developed using a single detection antibody. This multiplexing approach allowed up to 10 pairs of capture and detection antibody to be evaluated in parallel in a single well.

FIG. 1B provides heat maps displaying the ratio of signal to blank (S/B) achieved with each antibody pair. The figure also groups and color-codes antibodies based on the specificity of the antibodies for different LAM epitopes (FIG. 1C) as characterized using glycan arrays ((Choudhary et al. 2018) and FIG. 2B). Many antibody pairs showed high reactivity to purified LAM, but were relatively poor at detecting urinary LAM. In particular, only two antibodies (A194-01 and 27D2) were useful as detection antibodies for detecting LAM in urine. A194-01 antibody was the more sensitive of the two giving 2 to 5-fold higher signals in patient urine. Both of these antibodies target linear tetra-arabinoside (Ara4) or branched hexa-arabinoside (Ara6) structures in the arabinan domain of LAM. The specificity of 27D2 towards Ara6 confirmed the utility of synthetic LAM glycans fragments coupled to BSA as immunogens for the development of antibodies with specificity for defined LAM epitopes. Surprisingly, one antibody (MoAb2) provided high signals when used as a detection antibody for measuring purified LAM from cultured Mtb, but not when measuring urinary LAM. The glycan array studies indicate that this antibody targets LAM epitopes, the di- or tri-mannoside caps (Man2 or Man3), that are relatively specific to Mtb (Mishra et al. 2011; Chan et al. 2015). Without wishing to be limited to a single hypothesis, it is possible that Manp (Man1, Man2, Man2) caps without MTX are not stable in urine, for example because they might be degraded (e.g. by enzymes or another mechanism) and therefore these epitopes are not available in most urine samples.

Differences in relative reactivities to LAM from culture and urine were also observed for capture antibodies. Nearly all capture antibodies provided high signals for purified LAM from cultured Mtb when combined with A194-01 as the detection antibody, including antibodies targeting the branched Ara4 and Ara6 structure (A194-01, 27D2, 13H3), the Man2 or Man3 caps (MoAb3 and MoAb2) and Man2 or Man3. In agreement with its behavior as a detection antibody, when MoAb2 (which primarily targets Man2 and Man3 caps) was used as a capture antibody it also provided no or very low signals for urinary LAM. MoAb1 that targets Manp-MTX provided the highest signals in Urine from TB+/HIV− individuals. Without wishing to be limited to a single hypothesis, it is possible that the MTX motif protects Manp from degradation resulting in the presence of these epitopes in patient urine. As a resulting MoAb1 is an attractive, very reactive and specific capture antibody. To efficiently compare the clinical potential of these binding reagents and to get an increased understanding of the abundance of LAM structures in urine, eight pairs were developed and evaluated using a multiplexed panel of four capture antibodies covering a range of epitope specificities (13H3, 27D2, MoAb 1, MoAb3) and two detection antibodies (A194-01 and 27D2).

Analytical Assay Performance

FIG. 2A shows the calibration curve created by running 8 levels of purified LAM for the best performing antibody pair (MoAb1 as capture combined with A194-01 as detection antibody). The curve plots the assay signal as a function of LAM concentration. The intra-plate coefficients-of-variation (CVs) for the blank (no LAM) sample was <15% for all four capture antibodies with A194-01 as a detection antibody (Table 1A).

TABLE 1A
Results Using A194-01 As Detection Antibody

| | | | | Est. LOD |
|---|---|---|---|---|
| | Blank Signal | | Cal Signal | [LAM] (pg/mL) |
| Capture Ab | ECL | ECL CV | ECL CV | at S/B = 1.375 |
| 13H3 | 1,128 | 7% | 3% | 210 |
| 27D2 | 6,492 | 6% | 4% | 2,500 |
| MoAb1 | 264 | 12% | 3% | 11 |
| MoAb3 | 2,062 | 6% | 5% | 59 |

Table 1A. The table summarizes the analytical performance of four different anti-LAM capture antibodies when combined with the A194-01 detection antibody. The results were determined from 8 point calibration curves as described in FIG. 2A. The first two data columns show average signal and CV for the blank sample (n=14). The third shows the average CV for calibration standards providing signals above the detection threshold of 1.375-fold above the blank signal (based on 4 replicates per LAM concentration). The final column shows the estimates for LOD based on the LAM concentration expected to give a signal equal to the detection threshold as calculated by back-fitting to the best 4-PL fit to the calibration curve.

Based on these results, a signal of 37.5% above the blank (S/B=1.375) was defined as a signal threshold that was at least 2.5 standard deviations above the blank signal for all assay. CVs for the blank signals were dominated by the electronic noise of the system (±30 counts); as the signals increased above the blank signals, the CVs decreased considerably. On average, the CVs for LAM levels above this threshold were between 3 and 4% for all 12 pairs. Table 1A and FIG. 2A also provide the limits of detection (LODs) based on the signal threshold. Due to the higher signal to background ratios provided by the MoAb1 capture antibodies, this capture antibody provided more sensitive detection of purified LAM calibrators and LOD's of 11 pg/ml. When used as a detection antibody, 27D2 provided results that were highly correlated to results obtained using A194-10 (data not shown), but tended to provide lower signals and higher detection limits. Because of the high correlation and similar epitope specificities of the two antibodies, the analysis is focused on results obtained with the more sensitive A194-01 detection antibody. However 27D2 can be used as a replacement of A194-01 on the detector side.

Sample Preparation

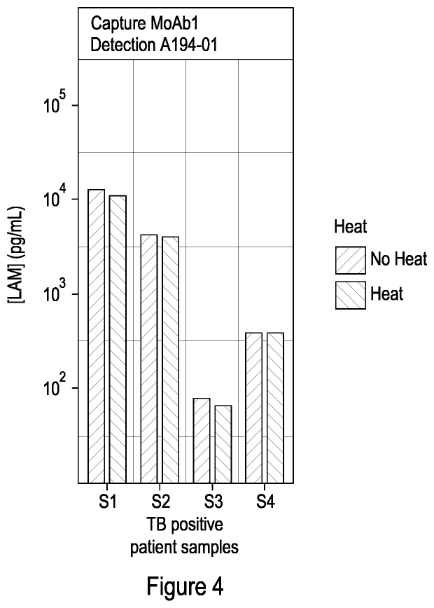

Prior to testing in the assay, urine samples were heat treated at 85° C. for 10 minutes to inactivate any anti-LAM antibodies that may be present in the samples. Little evidence was found that interfering antibodies were present in these samples, as signals were generally unchanged or only slightly increased by the heat treatment (FIG. 4). However, because the heat treatment did not appear to have any negative impact on LAM detection, it was decided to keep the heat treatment step in subsequent work.

To assess for matrix effects on assay performance, the following experiments were performed: (i) spike recovery experiments to show that LAM spiked into clinical urine samples provided the expected measured LAM levels, when compared to a calibration curve generated using LAM calibration standards prepared in a synthetic calibrator diluent (as in FIG. 2A) and (ii) dilution linearity experiments to show that the measured levels in urine samples decreased linearly with dilution of the urine into the calibrator diluent. The average recoveries for the spike and dilution experiments were in the accepted range of 80% to 120% for all the capture antibodies except for 27D2 (Table 1B). 27D2 had consistently low spike recoveries indicating that it may be subject to some degree of matrix interference that was not correctable by heating the samples.

TABLE 1B

| | | Capture Antibody | | | |
|---|---|---|---|---|---|
| Sample | Status | 13H3 | 27D2 | MoAb1 | MoAb3 |
| 1 | TB + HIV+ | 100% | 52% | 102% | 90% |
| 2 | TB + HIV+ | 106% | 56% | 102% | 95% |
| 3 | TB + HIV+ | 98% | 67% | 107% | 112% |
| 4 | TB − HIV− | 95% | 72% | 104% | 97% |
| 5 | TB − HIV− | 101% | 62% | 52% | 31% |
| 6 | TB − HIV− | 90% | 58% | 91% | 73% |
| 7 | TB − HIV− | 100% | 71% | 117% | 104% |
| Average | | 99% | 63% | 96% | 86% |
| 1 | TB + HIV+ | 107% | 104% | 105% | 126% |
| 2 | TB + HIV+ | 120% | 98% | 94% | 157% |
| 3 | TB + HIV+ | 94% | <LOD | 121% | 100% |
| 4 | TB + HIV+ | 88% | <LOD | 99% | 110% |
| 5 | TB + HIV− | 102% | 101% | 101% | 125% |

| | | | | |
|---|---|---|---|---|
| Average | 102% | 101% | 104% | 124% |

Table 1B shows spike recovery and dilution linearity for each of the four capture antibodies when paired with the A194-01 detection antibody. Spike recovery is the measured LAM concentration for purified LAM spiked into a urine sample, relative to the theoretical expected value. Each entry is the average recovery for three concentrations (300, 3,000 and 30,000 pg/ml) of LAM spiked into a sample. The recovery on dilution is the measured LAM concentration for a diluted LAM-positive urine sample relative to the expected value based on the measured urinary LAM in the undiluted sample (LAM levels in the undiluted samples ranged from roughly 1,000 to roughly 200,000 pg/mL). Each value is the average recovery for four dilutions ranging from 1:2 to 1:16. The table also shows the average values across all the tested samples.

Cross Reactivity

The LAM assays were tested for cross-reactivity against a panel of 10 different mycobacterium species and 20 different non-mycobacterial microorganisms that could potentially be present in urine samples. Table 2A provides the signal to blank ratios measured with each capture antibody when paired with the A194-01 detection antibody.

TABLE 2A

| Capture Antibody | | | |
|---|---|---|---|
| 13H3 | 27D2 | MoAb1 | MoAb3 |

Assay signal for blank sample

| | | | | |
|---|---|---|---|---|
| Blank | 1,897 | 13,744 | 235 | 5,414 |

Signal to Blank ratio (S/B) for 1:1,000 dilution of mycobacterium species

| | | | | |
|---|---|---|---|---|
| M. tuberculosis, H37Rv* | 1,342 | 439 | 1,977 | 99 |
| M. bovis* | 778 | 321 | 3,324 | 136 |
| M. fortuitum | 556 | 372 | ND | 691 |
| M. smegmatis | 4,033 | 669 | ND | 1,791 |
| M. abscessus | 37 | 23 | ND | 109 |
| M. chelonae | 9 | 5.2 | ND | 28 |
| M. gordonae* | 11 | 4 | 25 | 8 |
| M. intracellulare* | 3 | 3 | 129 | 18 |
| M. avium* | ND | ND | 1.4 | 3 |
| M. kansasii* | 4 | 2 | 10 | 7 |

Signal to Blank ratio (S/B) for 1:100 dilution of non-mycobacteria
(S/B > 1.375 for at least one assay)

| | | | | |
|---|---|---|---|---|
| Gordonia bronchialis | 2 | 9 | ND | 59 |
| Nocardia asteroides | 2 | 25 | ND | 62 |
| Rhodococcus sp. | 14 | 170 | ND | 359 |
| Tsukamurella paurometabolum | 26 | 57 | ND | 42 |

Organisms with S/B ≤ 1.375 at 1:100 Dilution for all Capture Antibodies
Candida albicans, Corynebacterium urealyticum, Escherichia coli,
Klebsiella pneumoniae, Streptococcus agalactiae,
Staphylococcus saprophyticus, Pseudomonas aeruginosa,
Staphylococcus aureus, Proteus mirabilis, Proteus vulgaris,
Neisseria gonorrhoeae, Haemophilus influenza, Enterococcus faecalis,
Enterobacter aerogenes, Chlamydia trachomatis

Table 2A shows the cross-reactivity of the LAM assays for a set of microorganisms. The results are provided for the indicated four capture antibodies when paired with the A194-01 detection antibody. The listed signal to blank (S/B) ratios were measured at a 1:10,000 dilution (mycobacterial samples) or 1:100 dilution (non-mycobacterial samples) of stock preparations obtained from ATCC or BEI. Data is only shown for organisms that gave S/B ratios greater than the assay threshold (1.375) for at least one capture antibody at the listed dilution. Microorganisms with undetectable cross-reactivity for all assay based on this threshold are listed at the bottom of the table. All the tested preparations were whole live cells except for Mtb and M. bovis (killed whole cell lysates) and M. smegmatis (PILAM purified from cell lysates). ND (not detectable) indicates that (i) the measured S/B ratio was less than 1.375 or (ii) the signal on the specific capture antibody spot was too low relative to the signals on the other spots (<0.2%) to accurately measure cross-reactivity. Slow growing mycobacteria are indicated with an asterisk*.

At the highest concentration tested (1:100 dilution of the stock ATCC or BEI materials), only four of the non-mycobacterial species provided S/B values greater than the assay threshold of 1.375 for at least one capture antibody (Nocardia asteroids, Gordonia bronchialis (Tsukamura), Rhodococcus sp., Tsukamurella paurometabolum). The strength of the cross-reactivity for these four species varied considerably across the different capture antibodies. 27D2 and MoAb3 showed the strongest cross-reactivities for all four species. 13H3 also cross-reacted with the four species, but had signals that were one to two orders of magnitude lower. MoAb1 provided the best discrimination and did not exhibit measurable cross-reactivity against any of the non-mycobacterial species at the tested concentrations.

As expected, all the capture antibodies provided strong signals for the TB-causing mycobacterial species Mtb and M. bovis; testing of 1:1,000 dilutions of these bacterial preparations gave signals that were above saturation levels for the assays. Large differences, however, were observed in the cross-reactivity of the different capture antibodies for other mycobacterial species tested at this dilution. All the capture antibodies except MoAb1 provided very high cross-reactivity for the fast-growing M. fortuitum and M. smegmatis species and provided saturating signals at the 1:1000 dilution. In contrast, when MoAb1 was used as the capture antibody, the cross-reactivity for these two species was at least three orders of magnitude lower than the other capture antibodies; and below the limit of detection. With the exception of M. intracellulare, MoAb1 also tended to have lower cross-reactivity for the other mycobacterium species, although the signals for these species tended to be lower and the differences between the capture antibodies tended to be smaller. Overall the Capture MoAb1/Detection A194-01 combination showed excellent specificity with no cross-reactivity to all non-mycobacteria microorganisms, now cross-reactivity to fast growing mycobacteria and only low-level cross-reactivity to slow growing Nontuberculous mycobacteria (at least 10-fold lower reactivity compared to Mtb).

Testing of Clinical Samples

Table 3A provides the characteristics of the study population.

TABLE 3A

| | | TB Negative | | TB Positive | |
|---|---|---|---|---|---|
| | All | HIV | HIV | HIV | HIV |
| | Subjects | Negative | Positive | Negative | Positive |
| Category | No. of Subjects | | | | |
| All subjects | 75 | 20 | 15 | 15 | 25 |
| Gender | | | | | |
| Female | 21 | 6 | 3 | 5 | 7 |
| Male | 49 | 9 | 12 | 10 | 18 |
| NA | 5 | 5 | 0 | 0 | 0 |
| Age | | | | | |
| 0 to 20 | 1 | 1 | 0 | 0 | 0 |
| 21 to 40 | 45 | 5 | 9 | 12 | 19 |
| 41 to 60 | 25 | 13 | 6 | 2 | 4 |
| 61+ | 2 | 1 | 0 | 1 | 0 |
| NA | 2 | 0 | 0 | 0 | 2 |
| Location | | | | | |
| Bangladesh | 5 | 5 | 0 | 0 | 0 |
| Peru | 19 | 3 | 14 | 2 | 0 |
| South Africa | 15 | 2 | 0 | 5 | 8 |
| Vietnam | 36 | 10 | 1 | 8 | 17 |
| CD4 Count | | | | | |
| <=100 cells/uL | 14 | 0 | 0 | 0 | 14 |
| >100 cells/uL | 8 | 0 | 0 | 0 | 8 |
| NA | 53 | 20 | 15 | 15 | 3 |
| Alere | | | | | |
| Negative | 62 | 20 | 15 | 13 | 14 |
| Positive | 13 | 0 | 0 | 2 | 11 |

Table 3A shows the characteristics of the study population broken down by TB and HIV status. NA indicates that information for the specified characteristic was not available for a study subject. CD4 cell counts were only available for TB/HIV+ subjects.

The samples were from FIND' s (Geneva, Switzerland) repository of TB clinical samples and were selected to include a range of geographical locations (Asia, Africa and S. America), and to cover the different combinations of TB and HIV status. CD4 counts were available for most of the TB+/HIV+ subjects and included subjects above and below the 100 cells/uL threshold used in the WHO algorithm for identifying immunocompromised patients most likely to benefit from the Alere LF-LAM test. In agreement with clinical studies of the clinical performance of the Alere test, the sensitivity of the Alere test for this panel of urine samples was 44% (11/25) for HIV+ subjects, but only 13% (2/15) for HIV− subjects.

FIG. 3A is a heat map that shows the measured LAM concentrations for the full sample set as a function of TB and HIV status. The heat map compares the concentrations measured with the four capture antibodies when A194-01 was used as the detection antibody. All the captures showed measurable concentrations of LAM in most of the urine samples from HIV+/TB+ subjects, but only MoAb1 and 13H3 detected LAM in urine from a significant proportion of the HIV−/TB+ subjects. Of these two, only MoAb1 provided good discrimination of TB+ and TB− subjects, as 13H3 detected LAM or LAM related structures in urine from many of the TB− subjects. The differences in performance for the MoAb1 and the other three capture antibodies (13H3, 27D2, MoAb3) are shown more clearly in the scatter plots in FIGS. 3B and 3C. Qualitatively, the signals from 13H3 and MoAb1 for samples from TB+ donors were well-separated from the assay threshold. The behavior of these two antibodies with TB− samples, however, was considerably different. 13H3 gave a wide spread distribution of signals for TB− samples. In contrast, the signals for TB− samples using the more TB-specific MoAb 1 capture antibody were tightly packed near the blank signal with the highest signal for a TB− sample having an S/B value of about 1.8 and all other samples providing signals below the LOD of 11 pg/ml. Color coding by Alere LF-LAM test result shows that the LAM signals detectable with the Alere test are one to two orders of magnitude above the detection limits for the immunoassay using the MoAb1 capture antibody, and that there were a number of samples from TB+ subjects that were detectable with the immunoassay, but not the Alere test, leading to a drastically increased sensitivity of the Capture MoAb1/Detector A194-01-based immunoassay.

Table 4 provides the measured sensitivity and specificity of the LAM assays for the test sample set. As an indicator of the separation between the assays signals for the TB− and TB+ groups, Table 4 also provides the area-under-curve (AUC) values from receiver operating curve (ROC) curve analysis.

TABLE 4

| | | Sensitivity | | | Specificity | | | |
|---|---|---|---|---|---|---|---|---|
| HIV | | Correct/ | | | Correct/ | | | |
| Status | Capture Ab | Total | % (95% CI) | | Total | % (95% CI) | | AUC (95% CI) |
| All | MoAb1 | 37/40 | 93% | (80%-97%) | 34/35 | 97% | (85%-100%) | 0.98 (0.95-1.00) |
| | 13H3 | 28/40 | 70% | (55%-82%) | 30/35 | 86% | (71%-94%) | 0.81 (0.71-0.91) |
| | 27D2 | 14/40 | 35% | (22%-50%) | 34/35 | 97% | (85%-100%) | 0.73 (0.62-0.85) |
| | MoAb3 | 21/40 | 53% | (37%-67%) | 28/35 | 80% | (64%-90%) | 0.73 (0.61-0.84) |
| HIV Neg. | MoAb1 | 12/15 | 80% | (55%-93%) | 20/20 | 100% | (84%-100%) | 0.95 (0.87-1.00) |
| | 13H3 | 6/15 | 40% | (20%-64%) | 17/20 | 85% | (64%-95%) | 0.60 (0.40-0.80) |
| | 27D2 | 2/15 | 13% | (4%-38%) | 19/20 | 95% | (76%-100%) | 0.50 (0.30-0.70) |
| | MoAb3 | 4/15 | 27% | (11%-52%) | 16/20 | 80% | (58%-92%) | 0.54 (0.34-0.74) |
| HIV Pos. | MoAb1 | 25/25 | 100% | (87%-100%) | 14/15 | 93% | (70%-100%) | 0.99 (0.97-1.00) |
| | 13H3 | 22/25 | 88% | (70%-96%) | 13/15 | 87% | (62%-96%) | 0.96 (0.90-1.00) |
| | 27D2 | 12/25 | 48% | (30%-67%) | 15/15 | 100% | (80%-100%) | 0.89 (0.80-0.99) |
| | MoAb3 | 17/25 | 68% | (48%-83%) | 12/15 | 80% | (55%-93%) | 0.88 (0.76-0.99) |
| All | Alere Test | 13/40 | 33% | (20%-48%) | 35/35 | 100% | (90%-100%) | 0.66 (0.59-0.74) |
| HIV Neg. | | 2/15 | 13% | (4%-38%) | 20/20 | 100% | (84%-100%) | 0.57 (0.48-0.66) |
| HIV Pos. | | 11/25 | 44% | (27%-63%) | 15/15 | 100% | (80%-100%) | 0.72 (0.62-0.82) |

Table 4 shows the accuracy of LAM assays using the selected panel of four capture antibodies (Abs) and A194-01 as a detection antibody compared to the Alere LF-LAM in the same sample set. The table provides the measured sensitivity (correctly classified TB+ samples/total number of TB+ samples) and specificity (correctly classified TB− samples/total number of TB− samples) for each of the four capture antibodies. The values were calculated for the full sample set (A11) or for the subsets of samples from HIV− and HIV+ subjects. 95% Confidence Intervals (CI) for the proportion were calculated using Wilson's method. The table also provides the AUC values from ROC analysis including confidence limits as determined by bootstrapping. For comparison, the bottom three rows present the analogous performance metrics for the Alere LF-LAM test for the same sample set.

Due to its combination of high signal for TB+ samples (including samples from HIV− subjects) and tight distribution of signals below the LOD for TB− samples, the AUC value for the assay using the MoAb1 capture antibody [0.98 (0.95-1.00)]—was significantly better than the AUC for the other capture antibodies. When examining only the HIV− samples, the difference between MoAb1 [0.95 (0.87-1.00)] and 13H3 [0.60 (0.40-0.80)] was even greater.

The AUC differences were reflected in the higher observed accuracy of the assay using MoAb1 [overall sensitivity=93% (80%-97%; 37/40), specificity=97% (85%-100%; 34/35) at a cut-off of 11 pg/ml], relative to 13H3 [overall sensitivity=70% (55%-82%, 28/40), specificity=86% (71%-94%); 30/35 at a cut-off of 210 pg/ml)]. The assay using the MoAb1 capture antibody was about 3 time more sensitive than the Alere LF-LAM assay [overall sensitivity=33% (20-48%; 13/40), specificity 100% (90-100%; 35/35)] while maintaining high specificity. The assay using the MoAb1 capture antibody was perfect in identifying TB+/HIV+ samples [MoAb1 sensitivity=100% (87%-100%; 25/25)].

FIG. 5 shows the associations of assay signal (for the assay using the MoAb1 capture antibody and the A194-01 detection antibody) with CD4 counts and Alere LF-LAM test results. HIV+ subjects that were strongly immunosuppressed (CD4<100 cells/μl) had significantly higher levels of LAM than HIV− subjects (FIG. 5B). The increased LAM levels appeared to correlate with immunosuppression as there was no significant difference between HIV+ subjects with CD4 counts>100 cells/μL and HIV− subjects. Confirming the qualitative picture from FIG. 3B, FIG. 5A shows that high Alere LF-LAM grade is associated with very high assay signals. The figure also highlights the significant number of TB+ subjects that have low but detectable signals by the immunoassay, but are undetected with the Alere test.

Additional Support for Specificity of the MoAb1 Capture Antibody

While the observed sensitivity and specificity of the MoAb1/A194-01 antibody pair was excellent, the signals observed for TB+/HIV− subjects tended to be low and near the assay threshold: urine from TB+/HIV− subjects provided a median S/B value of only 2.1. Additional experiments were to generate greater confidence that these signals reflect the result of a specific binding event and are not a result of variations in non-specific assay background.

In the first set of experiments, it was confirmed that concentrating LAM in urine samples generated a corresponding increase in the assay signals. Table 2B shows the effect of concentrating 7 urine samples with low levels of LAM to one fifth of their original volume using a centrifugal ultrafiltration device (Amicon) with a 10 kD molecular weight cut-off.

TABLE 2B

| | | [LAM] (pg/mL) on MoAb1 Spot | | |
|---|---|---|---|---|
| Sample | Status | 1X Sample | 5X Concentrate | Ratio |
| 1 | TB − HIV− | ND | ND | — |
| 2 | TB − HIV− | ND | ND | — |
| 3 | TB − HIV− | ND | ND | — |
| 4 | TB − HIV− | ND | ND | — |
| 5 | TB − HIV− | ND | ND | — |
| 6 | TB − HIV− | ND | ND | — |
| 7 | TB − HIV− | ND | ND | — |
| 8 | TB + HIV− | 52 | 229 | 4.4 |
| 9 | TB + HIV− | 29 | 93 | 3.2 |
| 10 | TB + HIV− | 49 | 232 | 4.7 |
| 11 | TB + HIV+ | 42 | 147 | 3.5 |
| 12 | TB + HIV+ | 43 | 147 | 3.4 |
| 13 | TB + HIV+ | 42 | 141 | 3.4 |
| 14 | TB + HIV+ | 74 | 360 | 4.9 |

Table 2B shows the results after urine samples with no detectable LAM (from TB−HIV− subjects) or low levels of detectable LAM (from TB+HIV− and TB+HIV+ subjects) were assayed for LAM using the MoAb1-A194-01 antibody pair. The samples were measured without concentration (1× Sample) or after 5-fold concentration using a centrifugal ultrafiltration device with a 10 kD cut-off (5× Concentrate). The sample pre-treatment step (85° C., 10 min.) was carried out prior to concentration. “ND” indicates that the assay signal was below the detection threshold for the assay. The Ratio column provides the ratio of the measured concentrations of LAM in the 5× concentrate to the 1× sample.

The concentration of the samples led to increases in the measured LAM concentration of between 3.2- and 4.9-fold, which approaches the expected theoretical 5-fold increase. In contrast, when samples from TB− donors with undetectable LAM levels underwent the same concentration process, the levels remained undetectable, so simple concentration of negative urine was not sufficient to generate this effect. A similar experiment was performed on a subset of the LAM-positive samples using a device with a 100 kD cut-off and determined that roughly 90% of the LAM passed into the filtrate (data not shown). This result suggests the measured species is between 10 and 100 kD in molecular weight and is consistent with previous studies characterizing the molecular weight of LAM from Mtb (Venisse et al. 1993).

In addition, it was confirmed that immobilized MoAb1 could be used to deplete LAM from urine samples. The depletion experiments were carried out in MSD large spot streptavidin plates coated with biotin-labeled MoAb1 to provide a high surface area of the antibody. Heat-treated samples were incubated in these wells (1 hr at room temperature with shaking) prior to transferring the samples to LAM assay plates to measure the remaining LAM levels. As shown in Table 3B, applying this depletion protocol to 10 urine samples from TB+ subjects having a wide range of LAM levels led to a median decrease in LAM levels of 56% (IQR: 47%-61%) relative to a sample that did not undergo depletion.

TABLE 3B

MoAb1 Spot

% Depletion

| Sample | Status | [LAM] (pg/mL) | Sham | MoAb1 |
|---|---|---|---|---|
| 1 | TB+/HIV− | 141 | 2% | 58% |
| 2 | TB+/HIV− | 610 | 3% | 45% |
| 3 | TB+/HIV− | 804 | 3% | 67% |
| 4 | TB+/HIV− | 307 | 2% | 30% |
| 5 | TB+/HIV+ | 63,060 | 2% | 30% |
| 6 | TB+/HIV+ | 781 | 2% | 75% |
| 7 | TB+/HIV+ | 5,539 | 1% | 56% |
| 8 | TB+/HIV+ | 424 | −4% | 56% |
| 9 | TB+/HIV+ | 114 | 19% | 62% |
| 10 | TB+/HIV+ | 296 | 1% | 52% |

Table 3B shows the results after heat treated urine samples with a range of LAM levels (from both TB+/HIV− and TB+/HIV+ subjects) were incubated in the wells of an MSD large spot streptavidin plate coated with biotin-labeled MoAb1 for one hour at room temperature with shaking to attempt to deplete LAM from the samples. As a control, the samples were also incubated in wells that were not coated with the antibody (the “Sham” condition). LAM levels were then measured in the depleted samples, as well as in the original undepleted samples, using the multiplex LAM assay with A194-10 as the detection antibody. The reported LAM concentrations are for the undepleted samples. The table also reports the percent reduction in the measured LAM concentration (% Depletion) for samples that were depleted using MoAb1 or using the Sham condition.

The presence of MoAb1 in the depletion step was required, as a sham condition where the depletion protocol was carried out in the absence of the antibody only provided a median decrease in LAM levels of 2% (IQR: 1%-3%).

Discussion

This case-control study sought to determine whether LAM is detectable in the urine of HIV−/TB+ and HIV+/TB+ patients using a highly sensitive ECL immunoassay with an LOD in the femtomolar (fM) range. The study employed a multiplexed format to enable the simultaneous evaluation of multiple antibodies of different specificities, to characterize how antibody specificity affects clinical performance.

Results of the ECL assay using the best performing pair of monoclonal antibodies (Capture MoAb1/Detector A194-01) showed almost 3-fold higher sensitivity and statistically indistinguishable specificity for tuberculosis case detection compared to the Alere LF-LAM in a small set of 75 urine samples from four countries collected from well-characterized patients with presumptive TB. All the HIV+ and a significant fraction of HIV− patients had detectable LAM concentrations above the assay detection limit of 11 pg/ml (0.6 pM). The detection limit of the assay was 25 to 50-fold below the cut-off of the Alere LF-LAM test which lies in the range of 250 to 500 pg/ml (Nakiyingi et al. 2014; Savolainen et al. 2013) and fails to detect TB patients with lower LAM concentrations. The results suggest that improvements in analytical sensitivity for detection of LAM can directly lead to improvements in clinical sensitivity for diagnosing TB.

The key driver for the increased diagnostic sensitivity at nearly perfect specificity for the immunoassay was the identification of a unique pair of well-defined monoclonal antibodies with binding specificities to distinct LAM epitopes that are present in the urine of TB patients. In a screen of each possible pair-wise combination of a set of anti-LAM antibodies from different sources, many pairs were found that were able to detect purified LAM from Mtb culture, but only a small subset showed good sensitivity for detecting LAM or LAM-related structures in patient urine. The choice of detection antibody appeared to be especially important for sensitive detection of LAM in urine and two antibodies (A194-01 and, to a lesser extent 27D2) were identified that provided substantially better performance as detection antibodies than the other candidates. Epitope mapping of these antibodies showed that both of these antibodies target arabinan domains of LAM, including both the linear Ara4 and branched Ara6 (hexa-Araf) motifs, indicating that these structures are relatively abundant in the urine of TB patients. Antibody engineering studies of A194-01 have shown that conversion to a monomeric Fab structure results in a large loss of binding activity (Pinter 2017) suggesting that multivalent binding of the full IgG to two arabinan motifs within a LAM molecule may be important for binding. The ability to increase avidity via multivalent binding may in turn play a role in this antibody's unique utility as a detection antibody.

As most of the candidate antibodies worked reasonably well as capture antibodies for detecting LAM in urine, selection of an optimal capture antibody is primarily driven by antibody specificity. Antibodies targeting both the linear Ara4 and branched Ara6 motifs A194-01, Imm 27D2), when paired with A194-01, were all able to detect LAM in at least some urine samples. Of this set of arabinan-specific capture antibodies, 13H3 tended to have the highest signals for urine from TB+ subjects, but showed relatively low specificity (86%) with some TB− samples giving high signals above the blank signal. While it was possible to develop a LAM assay using the 13H3/A194-01 pair, the results suggest that the performance of urinary LAM assays using only antibodies targeting non-TB specific arabinan epitopes may ultimately be limited by cross-reactivity with urinary LAM from other sources such as non-TB mycobacteria (NTB) or related organisms of the actinomycetales order. In particular, the Ara6 structures are not unique to Mtb LAM and the cross-reactivity studies confirmed that the 13H3/A194-01 pair cross-reacts with the non-mycobacterial actinomycetes Nocardia, Goronia, Rhodococcus and Tsukamurella, which are all known to produce LAM with Ara6 structures (Mishra et al. 2011; Briken et al. 2004). It is likely that the polyclonal antibodies used in the Alere LF-LAM test and previous commercial ELISA tests have similar limitations. An attempt to improve an older commercial ELISA test, the Clearview TB test, by concentrating urine prior to analysis found that the sensitivity could be significantly improved, but also found a corresponding decrease in specificity (Savolainen 2013). Similarly, the need to reduce false positive results from the Alere LF-LAM test led the manufacturer to revise the reference card of the test towards a higher assay cut-off in 2014, which increased specificity but decreased sensitivity. Furthermore, the cross-reactivity of the Alere LF-LAM test to mouth-residing Actinomyces and Nocardia is likely the reason that the assay is not specific enough for LAM detection in sputum (Dheda et al. 2010). It was also found that the Alere LF-LAM test were susceptible to cross-reactivity from environmental sources of LAM such as biofilms in the tubing of plate washers, emphasizing both the importance of antibody specificity as well as the need to protect against potential sources of environmental interferents when using non-TB specific antibodies.

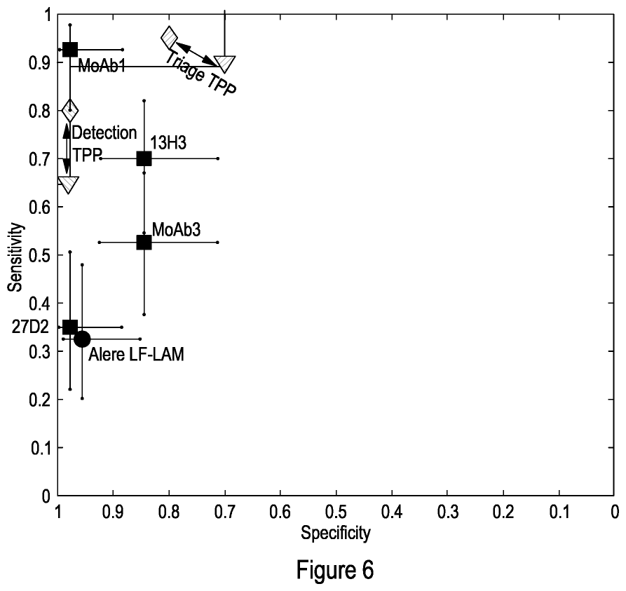

In addition to antibodies like 13H3 that target relatively non-specific LAM epitopes, capture antibodies targeting more TB-specific structures such as the Man2 and Man3 motifs (MoAb2), and the MTX-Man2 and MTX-Man3 motifs (MoAb1 ) were evaluated. Both provided strong signals for purified LAM from Mtb culture, but only MoAb1 detected LAM in urine samples from TB patients, indicating that a large fraction of any Man2 or Man3 cap motifs in urinary LAM must present the MTX modification. Surprisingly, the testing showed that pairing MoAb1 with the A194-01 detection antibody provided similar reactivity in the TB+ group like the 13H3/A194-01 pair, but that the MoAb1 capture antibody was able to achieve high clinical specificity. As shown in a graphical comparison of the observed clinical sensitivity and specificities of the different antibody pairs (FIG. 6), the MoAb1/A194-01 pair provided the best overall clinical sensitivity (93%) and specificity (97%). The high overall sensitivity largely reflected the excellent sensitivity of this pair for detecting LAM in urine from TB+HIV− subjects (80%). FIG. 6 also compares the observed performance of this pair to the WHO accuracy targets for POC TB tests and provides encouragement that the assay could meet the target specifications for POC TB tests for use in triage to identify patients for follow up TB testing, as well as the more stringent requirements for use in diagnosis.

Without wishing to be limited to a single hypothesis, it is possible that the epitope specificity of the MoAb1 capture antibody is responsible for its ability to provide sensitive detection while maintaining clinical specificity, as supported by the results of cross-reactivity testing of the MoAb1/A194-01 pair. No cross-reactivity was observed for the most common organisms responsible for urinary tract infections and, in contrast to the assay employing 13H3, no detectable cross-reactivity for the LAM-producing non-mycobacterial actinomycetes Nocardia, Goronia, Rhodococcus and Tsukamurella was observed. The MoAb1 capture also provided better discrimination of the TB-producing mycobacteria (Mtb and M. bovis) from most of the other mycobacteria species. In particular, no detectable cross-reactivity was observed with the fast-growing mycobacteria M. fortuitum, M. smegmatis, M. abscessus and M. chelonae, which produce LAM with little if any MTX modifications (Joe et al. 2006). In contrast, the 13H3 capture gave saturating or near saturating assay signals for the tested concentrations of M. fortuitum and M. smegmatis. A low level of cross-reactivity, for MoAb1, was observed with the slow growing NTM's M. gordonae, M. intracellulare, M. avium and M. kansasii, although the signals observed for these species were considerably lower than those measured for the TB− causing strains. It was also found that this pair was not susceptible to the unknown environmental LAM-like contaminant from the plate washers that generated signals for the Alere LF-LAM test. The TB-specificity of the MoAb1 antibody is also employed in an ELISA developed by Otsuka for LAM detection in sputum which, in contrast to the Alere LF-LAM test, does not cross-react with LAM produced by prevalent oral actinomycetes species (Kawasaki et al. 2018).

Although LAM was detectable in nearly all HIV-positive and HIV-negative patients using the MoAb1/A194-01 antibody pair, the study confirmed earlier findings of increased LAM concentrations in HIV-positive patients with low CD4 counts. Samples from TB/HIV co-infected patients with low CD4 counts ≤100 cells/μl had significantly higher LAM concentrations, with selected samples having >10 ng/ml LAM. Concentrations in samples from TB/HIV co-infected patients with high CD4 counts >100 cells/μl and TB/HIV-negative immunocompetent subjects were in the 11 to 1000 pg/ml range and lower (FIG. 5 and FIG. 3B). This effect is well known from large cohort studies with the Alere LF-LAM (Shah et al. 2016). Renal TB infection has been proposed as an explanation for high LAM concentrations in TB/HIV co-infected patients with low CD4 counts (Cox et al. 2015; Wood et al. 2012). The underlying mechanisms leading to LAM antigenuria in immunocompetent and HIV-negative patients of this study remains unclear. There is some evidence suggesting that LAM is actively secreted from infected alveolar macrophages (Strohmeier & Fenton 1999). Such an active process would be consistent with the important immunomodulatory properties of LAM that are likely to favor survival of TB in vivo (Lawn 2012). The process would also result in cell-free LAM or LAM fragments in the bloodstream which could potentially pass into urine through glomerular filtration. A study of LAM levels in serum and their correlation with urinary levels is currently in progress.

Summary of Findings

The improved performance of the assay, indicates that the development of an enhanced LAM-detection assays for TB diagnosis and screening in all HIV positive, but possibly also in HIV negative and immunocompetent patients, is well feasible. However, an assay with an LOD in the low picomolar or even femtomolar range and highly specific antibodies is required. Although the developed assay is highly sensitive and could provide a useful tool in a laboratory setting, it is not designed as a point-of-care test for use in typical primary care settings in low and middle income settings where laboratory facilities and trained personnel may not be available. Some of the most sensitive lateral flow assays reach sensitivities in the low pM range; for example a recently developed Malaria antigen detection assay reported a LOD of 2.5 pM HRP-2 (=80 pg/ml) which is close to the required analytical sensitivity (Das et al. 2017). Others proposed antigen concentration steps but assay complexity and cost could be a challenge. FIND and partners plan to transfer the findings from this study to a simple yet sensitive POC detection platform.

### EXAMPLE 2

### Epitope Mapping Performed with Antibodies

Epitope mapping was performed with MoAb1, MoAb2 and MoAb3.

Table 5 shows a description of the antibodies.

TABLE 5

| Antibody name | Specifications |
|---|---|
| MoAb1 | Higher affinity against Mtb LAM |
| | compared to LAM from |
| | Nontuberculous mycobacteria |
| | (NTM) |
| MoAb2 | Broadly reacts with |
| | mycobacterial LAM |
| MoAb3 | Broadly reacts with |
| | mycobacterial LAM |

2. Materials and Methods

The three antibodies (MoAb1, MoAb2, MoAb3) were analyzed to identify the oligosaccharide epitopes recognized by their binding sites. Epitope identification was carried out by measuring the binding of the antibodies to glycan arrays presenting a diverse set of 61 oligosaccharide structures (FIGS. 1D-1F). Briefly, oligosaccharide fragments were synthesized as previously described (Joe, M. et al. The 5-deoxy-5-methylthio-xylofuranose residue in mycobacterial lipoarabinomannan. Absolute stereochemistry, linkage position, conformation, and immunomodulatory activity. J. Am. Chem. Soc. 128, 5059-5072 (2006); Gadikota, R. R., Callam, C. S., Appelmelk, B. J. & Lowary, T. L. Synthesis of Oligosaccharide Fragments of Mannosylated Lipoarabinomannan Appropriately Functionalized for Neoglycoconjugate Preparation. J. Carbohydr. Chem. 22, 149-170 (2003); Joe, M., Bai, Y., Nacario, R. C. & Lowary, T. L. Synthesis of the docosanasaccharide arabinan domain of mycobacterial arabinogalactan and a proposed octadecasaccharide biosynthetic precursor. J. Am. Chem. Soc. 129, 9885-9901 (2007); Sahloul, K. & Lowary, T. L. Development of an Orthogonal Protection Strategy for the Synthesis of Mycobacterial Arabinomannan Fragments. J. Org. Chem. 80, 11417-11434 (2015); Zheng, R. B. et al. Insights into Interactions of Mycobacteria with the Host Innate Immune System from a Novel Array of Synthetic Mycobacterial Glycans. ACS Chem. Biol. 12, 2990-3002 (2017)).

The fragments were then conjugated to BSA and used to generate microarrays. Eight serial dilutions of antibodies (0.6 ng/ml, 2.4 ng/ml, 9.8 ng/ml, 39 ng/ml, 156 ng/ml, 625 ng/ml, 2.5 μg/ml, and 10 μg/ml) were incubated on the slides for 30 minutes at 37° C., and after washing, for 40 minutes stained with fluorescently labeled secondary anti-species antibodies (Cy™3 AffiniPure Goat anti-rabbit IgG from Jackson ImmunoResearch). After repeat washing and drying, florescence signals were measured using a GenePix 4000B scanner (Molecular Devices, Sunnyvale, USA) and intensity over background of each spot was quantified using ProMicroarray Image Analysis Software 6.1.

Results and Discussion

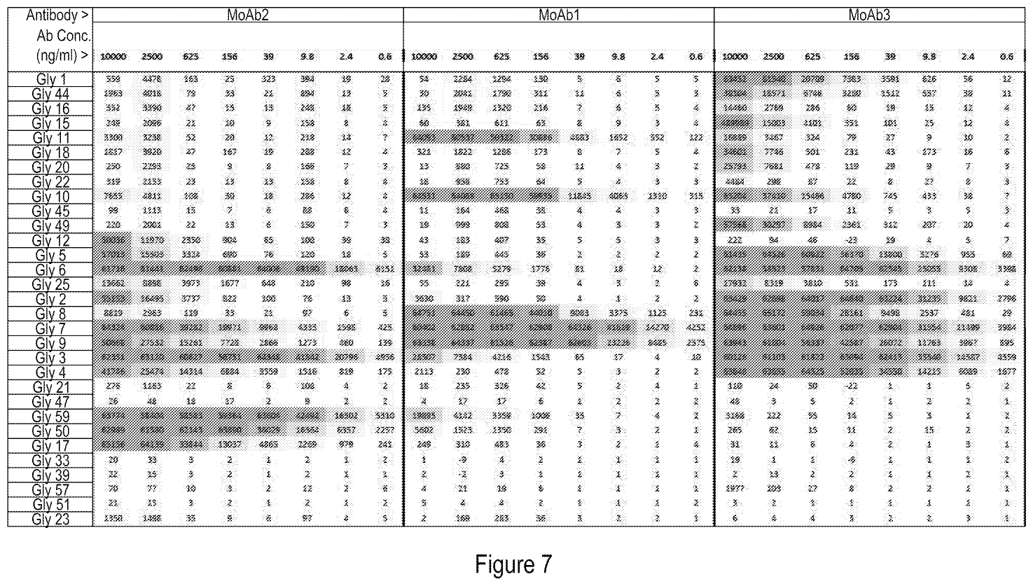

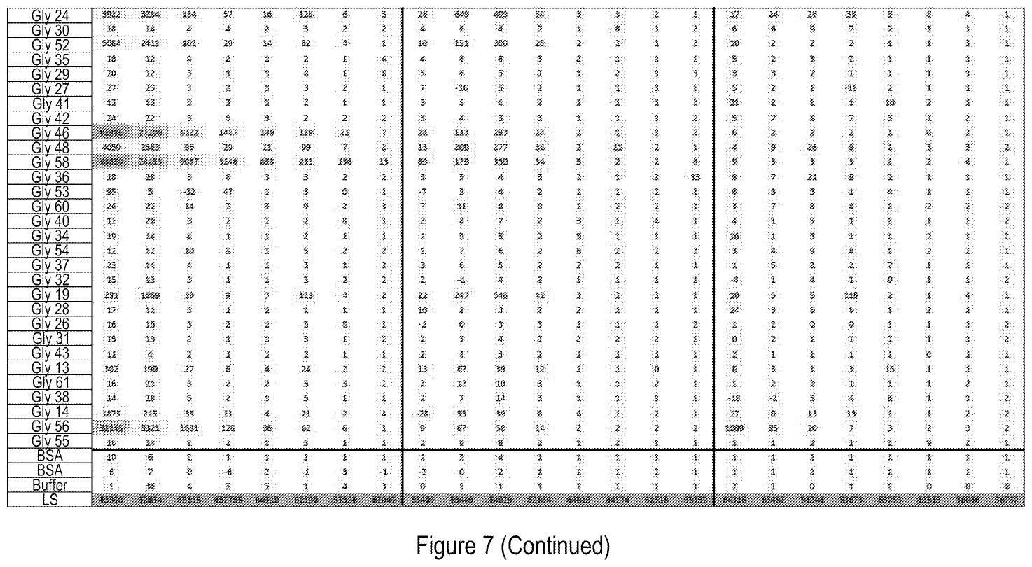

FIG. 7 shows the reactivity of the three monoclonal antibodies at eight different concentrations to all 61 oligosaccharide structures, three negative and one positive control spot.

MoAb2 preferentially recognized the dimannose-capped LAM with weak reactivity to mono- and tri substituted structures. This is consistent with the specificity of this antibody to Mtb, and the lack of reactivity with M. smegmatis PILAM (Glycan49). The reactivity for the dimannose-capped LAM was strongly inhibited by addition of MTX (compare reactivity of Glycan3 to Glycan 7), indicating that MoAb2 preferentially recognized the unmodified di-mannose glycan. The microarray analysis further showed that MoAb2 reacted strongly with several Manp-containing glycoconjugates that did not contain any Araf sugars. These epitopes shared a mannose structure with additional Manp residues linked either by α-(1→2) or α-(1→3) bonds to the end or middle Manp of this structure, and thus resembled both the mannan backbone and the capping structures. MoAb2 also reacted fairly well with Glycan59, a pentasaccharide structure which contained Manp-(1→3)-α-Manp linkages at both ends of the molecule. These results clearly indicate that this antibody solely depends on the Manp-containing component, and does not require any of the adjacent Araf residues for recognition. Manp is a conserved feature in Mtb and other slow growing mycobacteria, but does not occur in the capping fast growing species, such as M. smegmatis. However Example 1 from the testing in urine suggests that the unmodified Manp (i.e. the unmodified di-mannose glycan) is not available for binding in the urine of most TB patients. Without wishing to be limited by a single hypothesis, a possible reason is degradation of this epitope (e.g. by human enzymes like 1,2-mannosidases) in the absence of MTX. Therefore antibodies that target unmodified Manp motifs might not be diagnostically useful to detect ManLAM in the urine of patients.

In contrast, MoAb1 was uniquely specific for Manp-capped structures that were further substituted with an α-(1→4)-linked methylthio-xylose (MTX) residue. MoAb1 possessed the greatest reactivity with the MTX-modified dimannose (Glycan7) and trimannose (Glycan9) capped structures, and weaker reactivity with the MTX-modified mono-mannose structures (Glycan8, Glycan 10, Glycan 11). MoAb1 recognizes structures in which the MTX-Man motif was present on either the α-(1→5)-linked (Glycan10) or a-(1→3)-linked (Glycan11) arm of Ara6, suggesting that the poly-Araf structure may not be critical for recognition and that binding occurs primarily at the MTX-dimannose portion. The MTX substitution has been identified in all Mtb isolates analyzed to date and a recent report described a five-gene cluster dedicated to the biosynthesis of the MTX capping motif of Mtb LAM (Angala et al. 2017). M. smegmatis does also have all of these genes, so the lack of reactivity of MoAb1 with M. smegmatis and other fast growing mycobacteria is presumably related to the absence of Manp capping in PILAM, which precludes formation of this epitope. In contrast to MoAb2 MTX-Manp structures seem to be available for binding in the urine of TB patients. Without wishing to be limited by a single hypothesis, a possible explanation is that MTX protects the Manp units from degradation by enzymes or other degradation mechanisms that can take place in the body of a TB patient.

MoAb3 recognized uncapped Ara4 and Ara6 structures with low affinity and reacted most strongly with Ara4-Man1 (Glycan2), dimmannose capped Ara4 and Ara 6 structures (Glycan3 and Glycan6) and with MTX-modified Ara4-Man2 structure (Glycan7). Reactivity was lower with MTX-modified mono- and trimannose capped structures (Glycan8 and Glycan9). In contrast to MoAb2, MoAb3 did not recognize any of the polymannose structures (Glycan17, Glycan50, Glycan59), suggesting that the presence of the Ara structure(s) is important for the binding of MoAb3. In contrast to the other two antibodies MoAb3 had broader reactivity including weak binding to phospho-myo-inositol-capped Ara4 (Glycan49).

Table 6 shows a summary of the antibody binding results.

TABLE 6

| Antibody | Main Epitope | |
|---|---|---|
| name | Specificity | Comment |
| MoAb2 | Unmodified | Possible binding to mannan core and |
| | Man2 | caps. This type of LAM structures are |
| | | typically not present in TB patient |
| | | urine, which could be explained |
| | | through possible degradation of Man2 |
| | | caps by enzymes or other mechanisms |
| | | in the human body. |
| MoAb1 | MTX-Man2 | Binding is Independent of Ara. This |
| | | antibody is particularly important |
| | | for LAM detection in urine using |
| | | immunoassays. MTX seems to protect |
| | | Man2 from degradation in the human |
| | | body and i.e. in urine. |
| MoAb3 | Man1-Ara4 | Weak reactivity to uncapped Ara |
| | Man2-Ara4 | structures and PI-Ara4 |
| | Man2-Ara6 | |
| | MTX-Man2-Ara | |

Results Summary

Both, MoAb2 and MoAb1 are specific to epitopes that are only present in Mtb and other slow growing mycobacteria, but do not occur in fast growing mycobacteria, such as M. smegmatis or M. fortuitum, which explains the antibodies ability to distinguish slow from fast growing mycobacteria. It further explains the excellent analytical of assays based on these antibodies. Due to the absence of unmodified Man2 caps in urine of patients, MoAb2 seems not an important antibody for diagnostic immunoassays based on the detection of LAM in urine. In contrast MoAb1 detects MTX capped Manp structures which seem present and stable in urine. While MoAb3 shows overlapping reactivity to glycans that are also relevant for the two capture antibodies, the presence of Ara structures seem to be important for MoAb3 suggesting that the antibody binds a slightly different epitope.

### EXAMPLE

### Additional Data with Antibodies MoAb1 for Capture and A194-01 for Detection (Antibodies Under Test' or ‘Antibodies U. Test’ in the Figures)

This antibody combination was used to obtain further clinical data, indicating the efficacy of this antibody pair for detecting LAM in urine.

Methods

Study design and study population

Biobanked urine samples were assessed from inpatients (>18years) living with HIV, collected in three independent prospective cohort studies at two South African district hospitals. Criteria for the selection of a cohort were availability of frozen urine samples for a full cohort of hospitalized PLHIV in TB endemic settings, in whom a comprehensive work-up was performed to identify TB or alternative diagnoses. Standard national guidelines for TB and HIV management were used across all three cohorts. For the first cohort (“Cohort1”), adults with TB symptoms, able to produce sputum, were enrolled independent of CD4 count, on admission to Khayelitsha Hospital (KH) between February 2016 and August 2017. Cohortl excluded patients with extrapulmonary disease only. The second cohort (“Cohort2”) enrolled adults independent of CD4 counts who were admitted to adult medical wards at GF Jooste Hospital between June 2012 and October 2013, regardless of their ability to produce sputum or whether or not they reported TB symptoms.2,11 For Cohort2, study staff systematically attempted to collect urine, blood and two sputa for testing within 24 hours of admission. The third cohort (“Cohort3”) enrolled hospitalized PLHIV at KH with CD4≤350 cells/μl in whom TB was considered the most likely diagnosis at presentation between January 2014 and October 2016.

All cohorts excluded patients who were already receiving anti-TB therapy. In Cohorts 1 and 2, enrollment was done consecutively. Cohort3 used a random selection method after identifying all potentially eligible patients daily. In all cohorts, patients were enrolled at admission to hospital. Sputum, blood and urine specimens for M.tb reference standard testing were collected at enrollment and additional clinical samples were obtained during hospital admission and at follow-up. Sputum collection across cohorts was done by an experienced nurse or trained clinical research worker and sputum induction was performed, when required, as described previously.

All study-related activities were approved by the Human Research Ethics Committee (HREC) of the University of Cape Town (UCT). Written informed consent was obtained from patients, as per study protocols. Study participation did not affect standard of care. Reporting followed STARD guidelines. Retrospective urine LAM testing was supervised by the sponsor, FIND, and was performed at UCT in April 2018.

Laboratory Methods

Frozen urine aliquots of unprocessed urine were thawed to ambient temperature and mixed manually. Samples that were not immediately used for testing were stored at 4° C. for a maximum of 4 hours. Alere LF-LAM testing was performed following manufacturer's instructions. The pair of Antibodies Under Test, as described above (antibodies MoAb1 for capture and A194-01 for detection) were tested as follows. Briefly, urine was added to a reagent tube containing gold-labelled A194-01 up to the indicator line (approximately 200 μl), mixed, and incubated for 40 minutes at ambient temperature. After mixing again, two drops of urine/reagent from the tube were added to the test strip that is a lateral flow point-of-care immunoassay with the MoAb1 capture antibody immobilized on the ‘test’ line. In case the antigen is present in the urine, the MoAb1 capture antibody captures the antigen-A194-01-gold complex on the ‘test’ line to form the MoAb1-antigen-A194-01-gold complex in a sandwich format. The result was read within 10 minutes. No reading device is required, and the result was interpreted thorough visual inspection. Any line seen on the ‘test’ line was deemed TB positive.

Both assays were independently read by two readers blinded to the test results of the index or comparator test, respectively, the patient status and all other test results. After the initial independent test interpretation, readers compared results and, in case of discordance, re-inspected the test strip to establish the final consensus result (by mutual agreement) that was used for analysis. In case of assay failure, the test was repeated once.

For reference standard testing, the specimens were processed using standardized protocols in centralized accredited laboratories of the South African National Health Laboratory Service. Reference standard testing was performed on all available sputum specimens and included GeneXpert MTB/RIF (Xpert, Cepheid, Sunnyvale, USA; testing predates rollout of Xpert Ultra MTB/RIF), smear fluorescence microscopy after Auramine O staining, MGIT liquid culture (Becton Dickinson, Franklin Lakes, USA) and solid culture on Löwenstein-Jensen (LJ) medium.

The presence of Mtb complex in solid and liquid culture was confirmed with MPT64 antigen detection and/or the MTBDRplus line probe assays (Hain Lifesciences, Nehren, Germany). Blood cultures from all participants were done in BACTEC™ Myco/F Lytic culture vials (Becton Dickinson, Franklin Lakes, USA) and WHO prequalified in-vitro diagnostic tests were used for HIV testing (rapid diagnostic tests) and CD4 cell counting (flow cytometry). For urinary Xpert testing, 20-40 ml urine was centrifuged and following removal of supernatant the pellet was re-suspended in the residual urine volume and 0.75 ml was tested using Xpert.2 For Cohorts 2 and 3, additional respiratory and non-respiratory samples such as pleural fluid, cerebrospinal fluid and tissue fine needle aspirates were obtained, where clinically indicated, and tested using MGIT culture or Xpert. Clinical information, results from the pair of Antibodies Under Test and Alere LF-LAM results were not available to the assessors of the reference standard at the time of testing.

Reference standard categories

Patients were assigned to four diagnostic categories using a combination of clinical and laboratory findings. This was done by clinical investigators blinded to index test results prior to data analysis. “Definite TB” included patients with microbiologically confirmed Mtb (any culture or any Xpert MTB/RIF (“Xpert”) positive for Mtb during admission). “Not-TB” were patients with all microscopy, cultures and Xpert tests negative for Mtb (and at least one non-contaminated culture result), who were not started on anti-TB treatment and were alive or improved at three months follow-up. “Possible TB” were patients who did not satisfy the criteria for “Definite TB” but had clinical/radiological features suggestive of TB and were started on TB treatment. Patients that did not fall into any of these categories were deemed to be “unclassifiable” for this diagnostic accuracy study and removed from the main analyses. In a sensitivity analysis, the “unclassifiable” category was included to assess the impact of exclusions on diagnostic accuracy.

Statistical analysis

Accuracy (sensitivity, specificity, positive predictive value (PPV), negative predictive value (NPV), positive likelihood ratio (LR+) and negative likelihood ratio (LR−)) of the LFA with the pair of Antibodies Under Test and Alere LF-LAM was determined by comparison with a microbiological reference standard (MRS) as well as a composite reference standard (CRS). “Definite TB” versus “Not-TB” was used to allocate patients into reference standard positive versus negative groups. The “possible TB” group was deemed negative within an MRS but positive within a CRS. As per protocol, diagnostic accuracy was determined separately for each cohort. Heterogeneity was assessed using Cochran's Q-test.

To estimate pooled sensitivity and specificity across cohorts and CD4 strata, we performed a post-hoc analysis with a Bayesian bivariate random-effects model to account for study design differences. Results are presented with 95% confidence intervals (95% CI) based on Wilson's score method. The 95% CI of the differences (Δ) in percentages (for paired Antibodies Under Test and Alere LF-LAM) was computed using Tango's score method. The difference between two tests was considered to be significant if 0% was not included in the 95% CI of the difference. Cohen's kappa statistic was used to calculate agreement of positive and negative results between the two independent readers of the LAM tests. In an additional post-hoc analysis, the total number of microbiologically confirmed TB patients, (defined as the detection of Mtb by culture or Xpert in at least one clinical specimen of any type) was used as the denominator to calculate the comparative diagnostic yield of a single test of the Antibodies Under Test, Alere LF-LAM, sputum Xpert (cartridge version G4) and sputum smear microscopy test from samples collected within the first 24 hours of presentation. This analysis was restricted to Cohort2, as this cohort was designed to assess diagnostic yield by systematically attempting to collect these diagnostic samples (blood, urine and two sputum samples whenever possible) in all patients within the first 24 hours of admission. The data analysis was performed with R (version 3.5.1) and Matlab version 2017b.

Results

Patients

Overall, 1188 patients were eligible and considered for retrospective testing; 220 patients were excluded from the main analysis either due to unavailability of a urine sample (n=93), failed Antibodies Under Test tests (n=6) or being unclassifiable (n=121). The primary reasons for being “unclassifiable” were death before a diagnosis could be made (n=62) and loss to follow-up where a vital status or an improvement in clinical status was required for diagnosis (n=17). Of the 968 patients included in the main analysis, 600 (62%) were classified as definite TB, 91 (9%) as possible TB and 277 (29%) as not-TB. The microbiological reference standard for TB diagnosis was informed by a total of 6,397 culture and Xpert tests (on average 6.2/patient) and included 3,261 tests on sputum and 3,136 on non-sputum samples. A total of 236 patients (24.4%) could not provide a sputum sample. Definite TB diagnosis was based on the results from non-sputum samples for 19.5% of patients (117/600). TB prevalence was 49%, 38%, and 82% for Cohort 1, 2 and 3, respectively. Most patients were young immunocompromised adults (median age: 35 years), with median CD4 count of 113 cells/μl, 153 cells/μl, and 59 cells/μl for Cohort 1, 2 and 3, respectively. Across all cohorts, 45% had a history of prior TB treatment and all patients of Cohort 1 & 3 and 90% of Cohort 2 had a positive WHO symptom screen for TB.

Accuracy of Antibodies Under Test and Alere LF-LAM

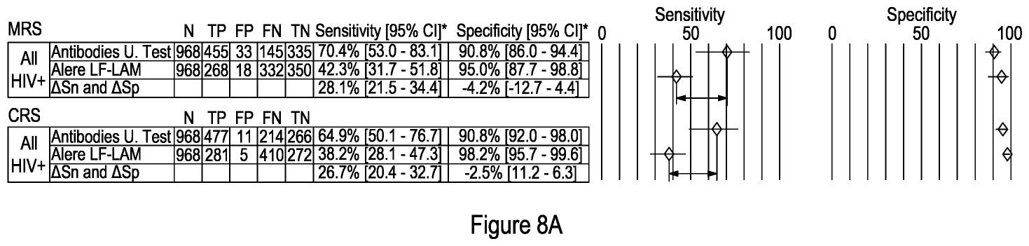

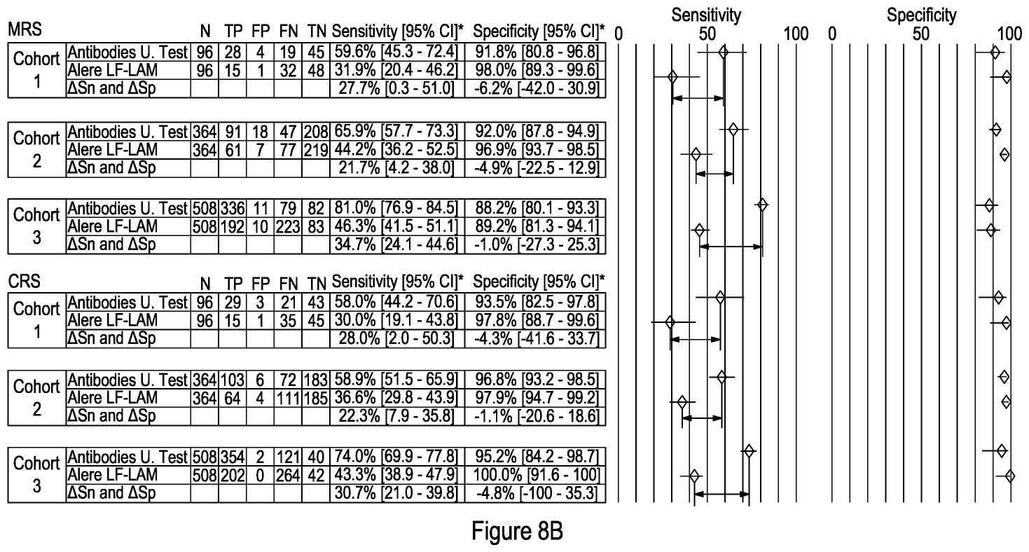

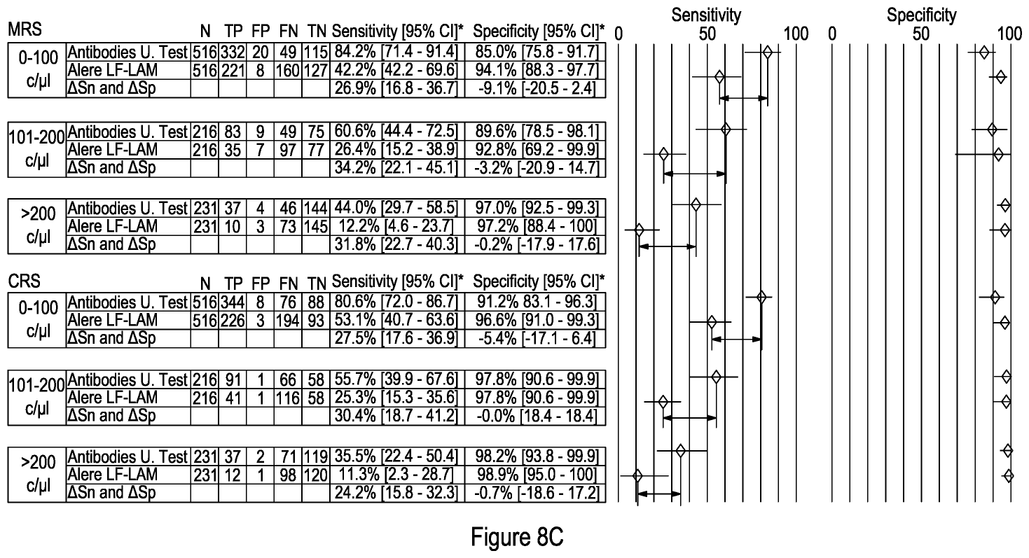

The analysis showed a sensitivity of 70⋅4% (95% CI: 53⋅0-83⋅1) for the Antibodies Under Test compared to 42⋅3% (31⋅7-51⋅8) for Alere LF-LAM against the MRS: a difference of 28⋅1% (21⋅5-34⋅4) between the two tests (FIG. 8). The highest Antibodies Under Test sensitivity against the MRS was observed in Cohort3 (81⋅0%), which enrolled patients with more advanced HIV-related immunosuppression (i.e. more patients with CD4 count below 100 cells/μl), compared to Cohort2 (65⋅9%) and Cohortl (59⋅6%). There is an inverse relationship of increasing sensitivity (for both assays) with decreasing CD4 counts when the patients were stratified by CD4 count. In patients with a CD4 count below 100 cells/μl, Antibodies Under Test had a sensitivity of 84⋅2% (71⋅4-91⋅4) compared to 57⋅3% (42⋅2-69⋅6) of Alere LF-LAM: a difference of 26⋅9% (16⋅8-36⋅7) between the two tests. A similar difference in sensitivity (31⋅8%; 22⋅7-40⋅3) was observed in more immunocompetent patients (CD4>200 cells/μl), but overall sensitivity was lower in this population for both assays; 44⋅0% 297 (29⋅7-58⋅5) for Antibodies Under Test and 12⋅2% (4⋅6-23⋅7) for Alere LF-LAM. Using the CRS, sensitivities of both assays were slightly lower: the overall Antibodies Under Test point estimate was 64⋅9% (50⋅1-76⋅7) and that of Alere LF-LAM 38⋅2% (28⋅1-47⋅3). Since the 95% confidence intervals of the differences in sensitivity between Antibodies Under Test and Alere LF-LAM did not overlap with zero (overall, in the three cohorts, and within CD4 strata), Antibodies Under Test was statistically considered to be significantly more sensitive than Alere LF-LAM (FIG. 8). Against the MRS, estimates of specificity were 90⋅8% (86⋅0-94⋅4) and 95⋅0% (87⋅7-98⋅8) for Antibodies Under Test and Alere LF-LAM respectively, representing a statistically non-significant difference (−4⋅2%; 12⋅7-4⋅4). Using the CRS, overall estimates of specificity were increased to 95⋅7% (92⋅0-98⋅0) for Antibodies Under Test and 98⋅2% (95⋅7-99⋅6) for Alere LF-LAM respectively, again representing a statistically non-significant difference (−2⋅5%; −11⋅2-6⋅3). Specificity of Antibodies Under Test assay was lower among those with CD4≤100 cells/μl (91⋅2% using the CRS) compared to those with CD4>100 cells/μl (FIG. 8). Eight of the 11 Antibodies Under Test false positive samples, using the CRS, were from patients with CD4 counts≤100 cells/μl. Using the CRS, the PPV for the three different cohorts ranged from 90⋅6-99⋅4% and 93⋅8-100⋅0% for Antibodies Under Test and Alere LF-LAM, respectively. The NPV ranged from 24⋅8-71⋅8% and 13⋅7-62⋅5% for Antibodies Under Test and Alere LF-LAM, respectively. Positive likelihood ratios (LR+) ranged from 8⋅9-18⋅5 and 13⋅8-17⋅3 for Antibodies Under Test and Alere LF-LAM, respectively. Negative likelihood ratios (LR−) ranged from 0⋅3-0⋅4 and 0⋅6-0⋅7 for Antibodies Under Test and Alere LF-LAM, respectively.

Diagnostic yield within 24 hours of admission

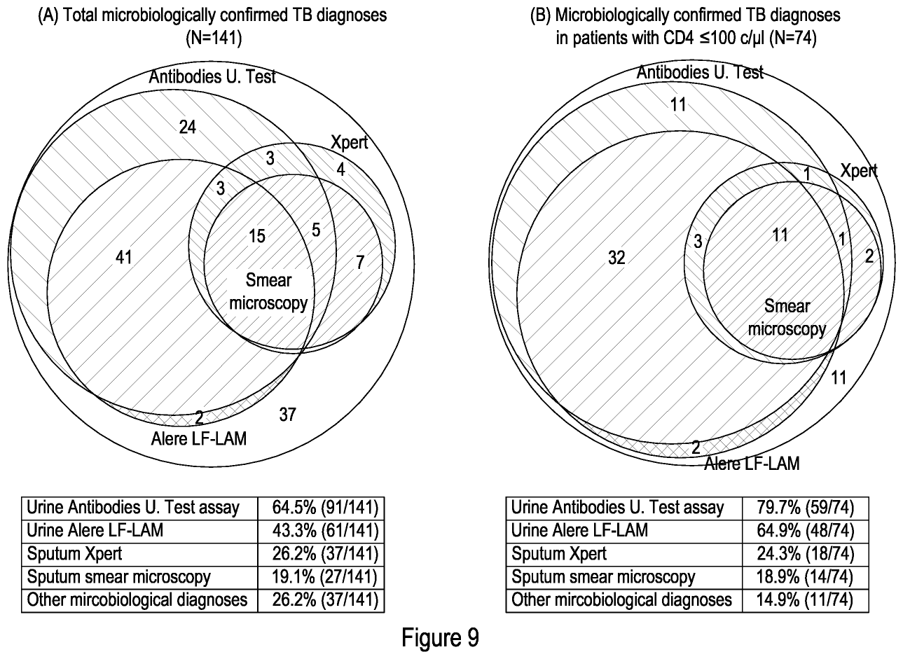

A total of 420 patients from Cohort2 were eligible for an analysis of diagnostic yield. Amongst eligible patients, only 36⋅4% (153/420) could produce a sputum sample within the first 24 hours of admission, whereas 99⋅5% (418/420) were able to provide a urine sample, as described previously for this cohort. A total of 141 patients had microbiologically confirmed TB. A total of 59⋅6% (84/141) of TB cases could be diagnosed on samples collected in the first 24 hours of admission using rapid tests: 26⋅2% (37/141) from sputum Xpert and 41⋅8% (59/141) from urine Xpert utilizing 1 ml of input volume. The remaining 40⋅4% (57/141) of TB diagnoses could not be achieved in the first 24 hours and were established by mycobacterial culture on any specimen collected at any point during patient admission, diagnosed by Xpert performed on concentrated samples from 20-40 ml urine and/or diagnosed by Xpert testing on specimens collected after the first 24 hours. The additional specimens collected for culture and Xpert testing included ascitic fluid, blood, urine, sputum, cerebrospinal fluid, gastric lavage, pus or pleural fluid). FIG. 9 illustrates the diagnostic yield of Antibodies Under Test and Alere LF-LAM compared to that of other rapid diagnostics from the first 24 hours. With the introduction of Antibodies Under Test, 64.5% (91/141) of TB cases could have been rapidly diagnosed at the bedside, within a few hours of presentation, compared to 43.3% (61/141) with Alere LF-LAM. A combination of sputum Xpert and Antibodies Under Test within the first 24 hours of admission would have been able to diagnose 72⋅3% (102/141) of microbiologically confirmed cases. A combination of sputum smear microscopy and Antibodies Under Test would have yielded 69⋅5% (98/141) of diagnoses.

Failure rate and reader agreement

In total, 1⋅6% (18/1095) Antibodies Under Test assays failed on the first attempt. Of the 15 that could be repeated, three failed on the second attempt resulting in a total overall error rate of 1⋅9% (21/1110 tests). Alere LF-LAM error rate was 0⋅4% (4/1095) on the first attempt and all four repeat tests delivered a result on the second attempt. In, sum the Antibodies Under Test assay has a similar failure rate like Alere LF-LAM and the Antibodies Under Test assay failure rate can be further reduced by standard quality control measures as they are commonly used in manufacturing of such assays. Agreement of two independent, blinded, visual reads of the same test was high for both Antibodies Under Test and Alere LF-LAM. Antibodies Under Test had an agreement rate of 97⋅0% (938/967; kappa coefficient 0⋅94), while Alere LF-LAM inter-reader agreement was 96⋅7% (934/966; kappa coefficient 0⋅92).

Discussion

In this assessment of 968 hospitalized PLHIV in a high-burden setting, the Antibodies Under Test point-of-care assay identified a significantly higher proportion of TB patients than Alere LF-LAM, while maintaining comparable specificity. In all sub-analyses, the sensitivity of Antibodies Under Test was significantly higher (range, 22-35%) than Alere LF-LAM. In patients with the highest risk of dying (patients with CD4≤100 cells/μl), Antibodies Under Test had the highest sensitivity of 84⋅2%, which was 26⋅9% higher than Alere LF-LAM. Combined with sputum Xpert, Antibodies Under Test could diagnose almost three-quarters of microbiologically-confirmed TB on the first day of hospitalization. The meta-analysis that formed the basis of the WHO recommendation for Alere LF-LAM reported an overall sensitivity of 45% in PLHIV which is similar to the Alere LF-LAM sensitivity of 42⋅3% seen in this study, suggesting that the population evaluated is similar to the populations in the WHO meta-analysis.

Collectively, these results suggest that if implemented in clinical practice and linked with appropriate treatment, the Antibodies Under Test point-of-care assay may be able to save lives by allowing for earlier diagnosis of HIV-associated TB in a large proportion of hospitalized patients. The point estimates of Antibodies Under Test specificity were lower compared to Alere LF-LAM. Although the differences in specificity between Antibodies Under Test and Alere LF-LAM were not significant, the reduced specificity, of both Alere LF-LAM and Antibodies Under Test, could be partially explained by an imperfect reference standard that lacks complete sensitivity. The existing reference standard is especially limited in its ability to identify TB in immunocompromised PLHIV, as these patients are more likely to have paucibacillary disease and/or extrapulmonary TB, making diagnosis more difficult.

It is possible that an imperfect reference standard could disproportionally affect a more sensitive test and result in increased false positives, i.e. lower specificity in this case of the more sensitive Antibodies Under Test assay. The decreasing specificity seen with decreasing CD4 count in this study and the improved specificity seen with the CRS in comparison to the MRS further supports this explanation. Cross-reactivity to common urinary tract pathogens and fast-growing non-tuberculous mycobacteria has been excluded in previous studies (Example 1, Table 2A) for the Antibodies Under Test.

### REFERENCES

Angala, S. kumar et al., 2017. Biosynthesis of the Methylthioxylose Capping Motif of Lipoarabinomannan in Mycobacterium tuberculosis ACS Chemical Biology, 12(3), pp.682-691. Available at: http://pubs.acs.org/doi/10.1021/acschembio.6b01071.

Briken, V. et al., 2004. Mycobacterial lipoarabinomannan and related lipoglycans: From biogenesis to modulation of the immune response. Molecular Microbiology, 53(2), pp.391-403.

Broger, T. et al., 2017. Diagnostic performance of tuberculosis-specific IgG antibody profiles in patients with presumptive tuberculosis from two continents. Clinical Infectious Diseases, 64(7).

Cazabon, D. et al., 2017. Quality of tuberculosis care in high burden countries: the urgent need to address gaps in the care cascade. International Journal of Infectious Diseases, 56, pp.111-116. Available at: http://linkinghub.elsevier.com/retrieve/pii/S1201971216312000.

Chan, C. E. et al., 2015. The diagnostic targeting of a carbohydrate virulence factor from M.Tuberculosis. Scientific Reports, 5, p.10281. Available at: http://www.nature.com/doifinder/10.1038/srep10281.

Cho, S. N. et al., 1990. Production of monoclonal antibodies to lipoarabinomannan-B and use in the detection of mycobacterial antigens in sputum. Yonsei medical journal, 31(4), pp.333-8. Available at: http://www.ncbi.nlm.nih.gov/pubmed/2127645.

Choudhary, A. et al., 2018. Characterization of the antigenic heterogeneity of lipoarabinomannan (LAM), the major surface glycolipid of Mycobacterium tuberculosis, and complexity of antibody specificities towards this antigen. Journal of Immunology, submitted.

Cox, J. a. et al., 2015. Is Urinary Lipoarabinomannan the Result of Renal Tuberculosis? Assessment of the Renal Histology in an Autopsy Cohort of Ugandan HIV-Infected Adults J. M. Sands, ed. PLOS ONE, 10(4), p.e0123323. Available at: http://dx.plos.org/10.1371/journal.pone.0123323.

Crawford, A. C. et al., 2017. Detection of the tuberculosis antigenic marker mannose-capped lipoarabinomannan in pretreated serum by surface-enhanced Raman scattering. The Analyst, 142(1), pp.186-196. Available at: http://xlink.rsc.org/?DOI=C6AN02110G.

Das, S. et al., 2017. Performance of a High-Sensitivity Rapid Diagnostic Test for Plasmodium falciparum Malaria in Asymptomatic Individuals from Uganda and Myanmar and Naive Human Challenge Infections. The American journal of tropical medicine and hygiene, 97(5), pp.1540-1550. Available at: http://www.ajtmh.org/content/journals/10.4269/ajtmh.17-0245.

Debad, J. D. et al., 2004. Clinical and Biological Applications of ECL. In Electrogenerated Chemiluminescence. Marcel Dekker, pp. 43-78.

Dheda, K. et al., 2010. Clinical utility of a commercial LAM-ELISA assay for TB diagnosis in HIV-infected patients using urine and sputum samples. B. Marais, ed. PLoS ONE, 5(3), p.e9848. Available at: http://dx.plos.org/10.1371/journal.pone.0009848 [Accessed Aug. 19, 2014].

Dorman, S. E. et al., 2017. Xpert MTB/RIF Ultra for detection of Mycobacterium tuberculosis and rifampicin resistance: a prospective multicentre diagnostic accuracy study. The Lancet Infectious Diseases, 3099(17), pp.1-9. Available at: http://linkinghub.elsevier.com/retrieve/pii/S1473309917306916.

Gadikota, R. R. et al., 2003. Synthesis of Oligosaccharide Fragments of Mannosylated Lipoarabinomannan Appropriately Functionalized for Neoglycoconjugate Preparation. Journal of Carbohydrate Chemistry, 22(3-4), pp.149-170. Available at: https://www.tandfonline.com/doi/full/10.1081/CAR-120021696.

Getahun, H. & Ford, N., 2016. Tackling the persistent burden of tuberculosis among people living with HIV. Journal of the International AIDS Society, 19(1), p.21002. Available at: https://www.ncbi.nlm.nih.gov/pubmed/27018421.

Hamasur, B. et al., 2015. A Sensitive Urinary Lipoarabinomannan Test for Tuberculosis. PLOS ONE, 10(4), p.e0123457. Available at: http://dx.plos.org/10.1371/journal.pone.0123457.

Joe, M. et al., 2007. Synthesis of the docosanasaccharide arabinan domain of mycobacterial arabinogalactan and a proposed octadecasaccharide biosynthetic precursor. Journal of the American Chemical Society, 129(32), pp.9885-9901.

Joe, M. et al., 2006. The 5-deoxy-5-methylthio-xylofuranose residue in mycobacterial lipoarabinomannan. Absolute stereochemistry, linkage position, conformation, and immunomodulatory activity. Journal of the American Chemical Society, 128(15), pp.5059-5072.

Kaur, D. et al., 2002. Characterization of the epitope of anti-lipoarabinomannan antibodies as the terminal hexaarabinofuranosyl motif of mycobacterial arabinans. Microbiology, 148(10), pp.3049-3057.

Kawasaki, M. et al., 2018. Lipoarabinomannan (LAM) in Sputum as a Potential Biomarker for Bacterial Load and Treatment Response in Pulmonary TB Patients. Journal of Clinical Microbiology, submitted.

Lawn, S. D., 2012. Point-of-care detection of lipoarabinomannan (LAM) in urine for diagnosis of HIV-associated tuberculosis: a state of the art review. BMC infectious diseases, 12(1), p.103. Available at: http://www.pubmedcentral.nih.gov/articlerender.fcgi?artid=3423001&tool=pmcentrez&renderty pe=abstract [Accessed Sep. 29, 2014].

Lawn, S. D. et al., 2011. Screening for HIV-associated tuberculosis and rifampicin resistance before antiretroviral therapy using the Xpert MTB/RIF assay: A prospective study. PLoS Medicine, 8(7).

Lawn, S. D. & Gupta-Wright, A., 2016. Detection of lipoarabinomannan (LAM) in urine is indicative of disseminated TB with renal involvement in patients living with HIV and advanced immunodeficiency: evidence and implications. Transactions of The Royal Society of Tropical Medicine and Hygiene, 110(3), pp.180-185. Available at: https://academic.oup.com/trstmh/article-lookup/doi/10.1093/trstmh/trw008.

Mishra, A. K. et al., 2011. Lipoarabinomannan and related glycoconjugates: structure, biogenesis and role in Mycobacterium tuberculosis physiology and host-pathogen interaction. FEMS Microbiology Reviews, 35(6), pp.1126-1157. Available at: http://femsre.oxfordjournals.org/lookup/doi/10.1111/j.1574-6976.2011.00276.x.

MSF & Stop-TB Partnership, 2017. Out of step 2017—TB Policies in 29 Countries, Available at: https://www.msfaccess.org/outofstep2017.

Nakiyingi, L. et al., 2014. Diagnostic Accuracy of a Rapid Urine Lipoarabinomannan Test for Tuberculosis in HIV-Infected Adults. JAIDS Journal of Acquired Immune Deficiency Syndromes, 66(3), pp.270-279. Available at: http://pubs.acs.org/doi/abs/10.1021/ac901991x.

Pai, M. et al., 2016. Tuberculosis. Nature Reviews Disease Primers, 2, p.16076. Available at: http://www.nature.com/articles/nrdp201676.

Paris, L. et al., 2017. Urine lipoarabinomannan glycan in HIV-negative patients with pulmonary tuberculosis correlates with disease severity. Science Translational Medicine, 9(420), p.eaa12807. Available at: http://stm.sciencemag.org/lookup/doi/10.1126/scitranslmed.aal2807.

Pereira Arias-Bouda, L. M. et al., 2000. Development of antigen detection assay for diagnosis of tuberculosis using sputum samples. Journal of clinical microbiology, 38(6), pp.2278-83. Available at: http://www.pubmedcentral.nih.gov/articlerender.fcgi?artid=86781&tool=pmcentrez&rendertype=abstract.

Peter, J. et al., 2010. Urine for the diagnosis of tuberculosis: current approaches, clinical applicability, and new developments. Current opinion in pulmonary medicine, 16(3), pp.262-70. Available at: http://www.ncbi.nlm.nih.gov/pubmed/20375787.

Peter, J. G. et al., 2016. Effect on mortality of point-of-care, urine-based lipoarabinomannan testing to guide tuberculosis treatment initiation in HIV-positive hospital inpatients: a pragmatic, parallel-group, multicountry, open-label, randomised controlled trial. Lancet, 387(10024), pp.1187-97. Available at: http://dx.doi.org/10.1016/S0140-6736(15)01092-2.

Sada, E. et al., 1992. Detection of lipoarabinomannan as a diagnostic test for tuberculosis. Journal of clinical microbiology, 30(9), pp.2415-8. Available at: http://www.ncbi.nlm.nih.gov/pubmed/1401008.

Sahloul, K. & Lowary, T. L., 2015. Development of an Orthogonal Protection Strategy for the Synthesis of Mycobacterial Arabinomannan Fragments. The Journal of Organic Chemistry, 80(22), pp.11417-11434. Available at: http://pubs.acs.org/doi/10.1021/acs.joc.5b02083.

Sarkar, P. et al., 2014. Application of lipoarabinomannan antigen in tuberculosis diagnostics: current evidence. Postgraduate Medical Journal, 90(1061), pp.155-163. Available at: http://www.ncbi.nlm.nih.gov/pubmed/24429376.

Savolainen, L. et al., 2013. Modification of clearview tuberculosis (TB) enzyme-linked immunosorbent assay for TB patients not infected with HIV. Clinical and vaccine immunology : CVI, 20(9), pp.1479-82. Available at: http://www.pubmedcentral.nih.gov/articlerender.fcgi?artid=3889579&tool=pmcentrez&renderty pe=abstract.

Shah, M. et al., 2016. Lateral flow urine lipoarabinomannan assay for detecting active tuberculosis in HIV-positive adults. In M. Shah, ed. Cochrane Database of Systematic Reviews. Chichester, UK: John Wiley & Sons, Ltd. Available at: http://doi.wiley.com/10.1002/14651858.CD011420.pub2.

Steingart, K. R. et al., 2014. Xpert® MTB/RIF assay for pulmonary tuberculosis and rifampicin resistance in adults. K. R. Steingart, ed. The Cochrane database of systematic reviews, 1(1), p.CD009593. Available at: http://doi.wiley.com/10.1002/14651858.CD009593.pub3.

Strohmeier, G. R. & Fenton, M. J. , 1999. Roles of lipoarabinomannan in the pathogenesis of tuberculosis. Microbes and Infection, 1(9), pp.709-717. Available at: http://linkinghub.elsevier.com/retrieve/pii/S1286457999800720.

Svenson, S., 1997. Patent WO1997034149: Method of diagnosing a mycobacterial disease and immunoassay kit. Available at: https://www.google.ch/patents/WO1997034149A1.

Uplekar, M. et al., 2015. WHO's new end TB strategy. Lancet (London, England), 385(9979), pp.1799-801. Available at: http://www.ncbi.nlm.nih.gov/pubmed/25814376.

Venisse, A. et al., 1993. Structural features of lipoarabinomannan from Mycobacterium bovis BCG. Determination of molecular mass by laser desorption mass spectrometry. The Journal of biological chemistry, 268(17), pp.12401-11. Available at: http://www.ncbi.nlm.nih.gov/pubmed/8509380.

Wood, R. et al., 2012. Lipoarabinomannan in urine during tuberculosis treatment: association with host and pathogen factors and mycobacteriuria. BMC Infectious Diseases, 12(1), p.47. Available at: http://www.biomedcentral.com/1471-2334/12/47.

World Health Organization, 2017. Global Tuberculosis Report 2017, Available at: http://apps.who.int/iris/bitstream/10665/259366/1/9789241565516-eng.pdf?ua=1.

World Health Organization, 2014. High-priority target product profiles for new tuberculosis diagnostics: report of a consensus meeting. (April). Available at: http://apps.who.int/iris/bitstream/10665/135617/1/WHO_HTM_ TB_2014.18 eng. pdf.

World Health Organization, 2015. The use of lateral flow urine lipoarabinomannan assay (LF-LAM) for the diagnosis and screening of active tuberculosis in people living with HIV. (November). Available at: http://www.who.int/tb/publications/use-of-lf-lam-tb-hiv/en/.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1.15. (canceled)

16. A method for differentially detecting a presence of disease-causing mycobacteria in a subject, comprising contacting
a first antibody, for the detection of an antigen associated with mycobacteria in an in vitro sample urine of a subject, wherein said antigen comprises ManLAM (Mannose capped Lipoarabinomannan), said first antibody specifically binding to said ManLAM molecules from said urine, wherein said first antibody binds to said ManLAM with an affinity having a KD of 3×10⁻⁸ M or less, and wherein said first antibody binds to LAM molecules that are not capped or that are capped with inositol phosphate with an affinity having a KD of 10⁻³ M or more; and
a second antibody for the detection of an antigen, wherein said second antibody does not bind to the same antigen as the first antibody, and wherein said antigens comprise said ManLAM molecules; wherein one of said first and second antibodies is a capture antibody and wherein the other of said first and second antibodies is a detection antibody in a sandwich immunoassay,
with the urine of the subject; detecting binding of one of said antibodies to one of said antigens in the urine; if said one antibody binds specifically to said one antigen in the urine with an affinity having a KD of 3×10⁻⁸ M or less, determining that said disease-causing mycobacteria characterized by said ManLAM molecules is present in the subject's body; wherein one of said antibodies binds to one of said antigens of said disease-causing mycobacteria in the urine with a signal at least three times greater than to an antigen of non-disease causing mycobacteria; wherein said second antibody is characterized as binding to poly-arabinose structures of said ManLAM molecules with an affinity having a KD of 3×10⁻⁵ M or less; wherein the detected ManLAM antigen specific sandwich immunoassay signal in a urine sample from a subject without tuberculosis is below 11 pg ManLAM/ml for at least 70% of the samples in a population; wherein the signal is below the limit of detection for at least 80% of the samples in the population; wherein the detected ManLAM antigen specific sandwich immunoassay signal in a urine sample from a subject with tuberculosis is above 11 pg ManLAM/ml for at least 40% of the samples in a population; and wherein the signal is above the limit of detection for at least 60% of the samples in the population.

17. (canceled)

18. (canceled)

19. (canceled)

20. The method of claim 1, wherein said antibody binds specifically to ara4 and/or ara6.

21. The method of claim 1, comprising contacting the urine with an antibody selected from the group consisting of MoAb1, 13H3, 27D2 and A194-01 antibodies.

22. The method of claim 21, comprising contacting the urine with a combination of a plurality of the MoAb1, 13H3, 27D2 or A194-1 antibodies in a sandwich immunoassay.

23. The method of claim 22, comprising contacting the urine with a combination of the MoAb1 and A194-1 antibodies in a sandwich immunoassay.

24. The method of claim 21, comprising applying the MoAb1 antibody to a sample with a suitable second antibody to achieve a fold change of 3 or greater between median signals of samples from subjects suffering from tuberculosis compared to samples from subjects without tuberculosis using a suitable reference standard diagnosis for classification of the subjects.

25. The method of claim 23, wherein said reference standard diagnosis is based on mycobacterial culture or PCR based methods to classify subjects.

26. The method of claim 21, comprising applying the MoAb 1 antibody to a sample to detect at least 20% more subjects suffering from tuberculosis compared to samples from subjects without tuberculosis using a suitable comparative standard assay, wherein said suitable comparative standard assay comprises the Alere LF-LAM.

27. (canceled)

28. (canceled)

29. The method of claim 1, wherein the signal is below the limit of detection for at least 90% of the samples in the population.

30. The method of claim 29, wherein the signal is below the limit of detection for at least 95% of the samples in the population.

31. The method of claim 30, wherein the signal is below the limit of detection for at least 97% of the samples in the population.

32. (canceled)

33. (canceled)

34. (canceled)

35. The method of claim 1, wherein the signal is above the limit of detection for at least 75% of the samples in the population.

36. The method of claim 35, wherein the signal is above the limit of detection for at least 90% of the samples in the population.

37. The method of claim 1, further comprising detecting TB disease-causing mycobacteria in the subject in the absence of the HIV virus.

38. The method of claim 1, wherein an AUC (area under the curve) of an immunoassay based on binding of said antibodies to said antigen is at least 0.70.

39. The method of claim 38, wherein said AUC is at least 0.80.

40. The method of claim 39, wherein said AUC is at least 0.85.

41. The method of claim 40, wherein said AUC is at least 0.90.

42. The method of claim 41, wherein said AUC is at least 0.95.

43. The method of claim 42, wherein said AUC is at least 0.98.

44. The method of claim 1, comprising applying a combination of the MoAb1 antibody or the 13H3 antibody as the first antibody, and the A194-01 antibody or the 27D2 antibody as the second antibody to detect an antigen associated with mycobacteria in an in vitro urine sample from a subject, in an immunoassay in which one of the first and second antibodies is the capture antibody and the other of the first and second antibodies is the detection antibody.

45. The method of claim 44, wherein said detection is performed by using an immunoassay, wherein the combination has at least 20% higher clinical sensitivity than the Alere LF-LAM test.

46. The method of claim 1, further comprising diagnosing the subject with tuberculosis according to a presence of said disease-causing mycobacteria in the body of the subject.

47. The method of claim 46, wherein said diagnosing further comprises detecting a presence of an active tubercular infection in the subject.

48. The method of claim 47, further comprising monitoring efficacy of treatment of the subject for tuberculosis according to the presence of said disease-causing mycobacteria.

49. The method of claim 1, further comprising concentrating said antigen comprising ManLAM in the sample prior to detection with the immunoassay to further increase clinical sensitivity.

50. The method of claim 49, wherein said concentrating said antigen comprises applying magnetic beads or ultrafiltration to the sample.

51. The method of claim 1, further comprising differentiating between a presence of a disease-causing mycobacteria in the subject and a non-disease causing mycobacteria in the subject.

52. The method of claim 1, further comprising specifically detecting a presence of a disease-causing mycobacteria in the subject in a presence of contaminating bacteria from an environment of the subject.

53. The method of claim 52, wherein said contaminating bacteria comprise one or more of Gordonia bronchialis, Nocardia asteroids, Rhodococcus sp., Tsukamurella paurometabolum, Candida albicans, Corynebacterium urealyticum, Escherichia coli, Klebsiella pneumoniae, Streptococcus agalactiae, Staphylococcus saprophyticus, Pseudomonas aeruginosa, Staphylococcus aureus, Proteus mirabilis, Proteus vulgaris, Neisseria gonorrhoeae, Haemophilus influenza, Enterococcus faecalis, Enterobacter aerogenes, or Chlamydia trachomatis, or Nontuberculous mycobacteria.

54. The method of claim 1, further comprising heating the urine before contacting said antibody.
